(19) United States
(12) Patent Application Publication
(10) Pub. No.: US 2007/0264348 A1
(43) Pub. Date: Nov. 15, 2007

(54) Nanoparticulate fibrate formulations

(75) Inventors: Tuula Ryde, Malvern, PA (US); Evan E. Gustow, Villanova, PA (US); Rajeev Jain, Collegeville, PA (US); Rakesh Patel, Bensalem, PA (US); Michael John Wilkins, Middleton (IE)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International, Ltd.

(21) Appl. No.: 11/710,607

(22) Filed: Feb. 26, 2007

Related U.S. Application Data
(63) Continuation-in-part of application No. 11/522,528, filed on Sep. 18, 2006, now abandoned.
Application No. 11/522,528 is a continuation of application No. 11/275,278, filed on Dec. 21, 2005, now abandoned.
Application No. 11/275,278 is a continuation-in-part of application No. 10/444,066, filed on May 23, 2003, now Pat. No. 7,276,249.
Application No. 10/444,066 is a continuation-in-part of application No. 10/370,277, filed on Feb. 21, 2003, now abandoned.
(60) Provisional application No. 60/383,294, filed on May 24, 2002.

(51) Int. Cl. A61K 31/19

(52) U.S. Cl. 424/489; 514/277; 514/396; 514/406; 514/460; 514/553; 514/570; 514/635; 514/675

(57) ABSTRACT

The present invention is directed to fibrate compositions having improved pharmacokinetic profiles and reduced fed/fasted variability. The fibrate particles of the composition have an effective average particle size of less than about 2000 nm.

### CROSS REFERENCE TO RELATED APPLICATIONS

This application is a continuation-in-part of U.S. patent application Ser. No. 11/522,528, filed on Sep. 18, 2006, which is a continuation of U.S. patent application Ser. No. 11/275,278, filed on Dec. 21, 2005, which is a continuation-in-part of U.S. patent application Ser. No. 10/444,066, filed on May 23, 2003, currently pending, which is a continuation-in-part of U.S. patent application Ser. No. 10/370,277, filed on Feb. 21, 2003, now abandoned, which claims priority of U.S. Provisional Application No. 60/383,294, filed on May 24, 2002.

### FIELD OF THE INVENTION

The present invention relates to a nanoparticulate composition comprising a fibrate, preferably fenofibrate or a salt thereof. The nanoparticulate fibrate, preferably fenofibrate, particles have an effective average particle size of less than about 2000 nm.

### BACKGROUND OF THE INVENTION

A. Background Regarding Nanoparticulate Compositions

Nanoparticulate compositions, first described in U.S. Pat. No. 5,145,684 (“the '684 patent”), are particles consisting of a poorly soluble therapeutic or diagnostic agent having adsorbed onto the surface thereof a non-crosslinked surface stabilizer. The '684 patent does not describe nanoparticulate compositions of a fibrate. Methods of making nanoparticulate compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using Iodinated Aromatic Propanedioates;” 5,336,507 for “Use of Charged Phospholipids to Reduce Nanoparticle Aggregation;”5,340,564 for “Formulations Comprising Olin 10-G to Prevent Particle Aggregation and Increased Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate;” 6,428,814 for “Bioadhesive Nanoparticulate Compositions Having Cationic Surface Stabilizers;” 6,431,478 for “Small Scale Mill;” 6,432,381 for “Methods for Targeting Drug Delivery to the Upper and/or Lower Gastrointestinal Tract,” 6,582,285 for “Apparatus for Sanitary Wet Milling,” 6,592,903 for “Nanoparticulate Dispersions Comprising a Synergistic Combination of a Polymeric Surface Stabilizer and Dioctyl Sodium Sulfosuccinate,” 6,656,504 for “Nanoparticulate Compositions Comprising Amorphous Cyclosporine and Methods of Making and Using Such Compositions,” 6,582,285 for “Apparatus for Sanitary Wet Milling;” 6,5-92,903 for “Nanoparticulate Dispersions Comprising a Synergistic Combination of a Polymeric Surface Stabilizer and Dioctyl Sodium Sulfosuccinate,” 6,742,734 for “System and Method for Milling Materials,” 6,745,962 for “Small Scale Mill and Method Thereof,” 6,811,767 for “Liquid droplet aerosols of nanoparticulate drugs,” 6,908,626 for “Compositions having a combination of immediate release and controlled release characteristics,” 6,969,529 for “Nanoparticulate compositions comprising copolymers of vinyl pyrrolidone and vinyl acetate as surface stabilizers,” 6,976,647 for “System and Method for Milling Materials,” 6,991,191 for “Method of Using a Small Scale Mill,” 7,101,576 for “Nanoparticulate Megestrol Formulation,” all of which are specifically incorporated by reference.

In addition, U.S. Patent Publication No. 20060246142 for “Nanoparticulate quinazoline derivative formulations,” U.S. Patent Publication No. 20060246141 for “Nanoparticulate lipase inhibitor formulations,” U.S. Patent Publication No. 20060216353 for “Nanoparticulate corticosteroid and antihistamine formulations,” U.S. Patent Publication No. 20060210639 for” Nanoparticulate bisphosphonate compositions,” U.S. Patent Publication No. 20060210638 for “Injectable compositions of nanoparticulate immunosuppressive compounds,” U.S. Patent Publication No. 20060204588 for “Formulations of a nanoparticulate finasteride, dutasteride or tamsulosin hydrochloride, and mixtures thereof,” U.S. Patent Publication No. 20060198896 for “Aerosol and injectable formulations of nanoparticulate benzodiazepine,” U.S. Patent Publication No. 20060193920 for “Nanoparticulate Compositions of Mitogen-Activated (MAP) Kinase Inhibitors,” U.S. Patent Publication No. 20060188566 for “Nanoparticulate formulations of docetaxel and analogues thereof,” U.S. Patent Publication No. 20060165806 for “Nanoparticulate candesartan formulations,” “U.S. Patent Publication No. 20060159767 for “Nanoparticulate bicalutamide formulations,” U.S. Patent Publication No. 20060159766 for “Nanoparticulate tacrolimus formulations,” U.S. Patent Publication No. 20060159628 for “Nanoparticulate benzothiophene formulations,” U.S. Patent Publication No. 20060154918 for “Injectable nanoparticulate olanzapine formulations,” U.S. Patent Publication No. 20060121112 for “Topiramate pharmaceutical composition,” U.S. Patent Publication No. 20020012675 A1, for “Controlled Release Nanoparticulate Compositions,” U.S. Patent Publication No. 20040195413 A1, for “Compositions and method for milling materials,” U.S. Patent Publication No. 20040173696 A1 for “Milling microgram quantities of nanoparticulate candidate compounds,” U.S. Patent Publication No. 20050276974 for “Nanoparticulate Fibrate Formulations”; U.S. Patent Publication No. 20050238725 for “Nanoparticulate Compositions Having a Peptide as a Surface Stabilizer”; U.S. Patent Publication No. 20050233001 for “Nanoparticulate Megestrol Formulations”; U.S. Patent Publication No. 20050147664 for “Compositions Comprising Antibodies and Methods of Using the Same for Targeting Nanoparticulate Active Agent Delivery”; U.S. Patent Publication No. 20050063913 for “Novel Metaxalone Compositions”; U.S. Patent Publication No. 20050042177 for “Novel Compositions of Sildenafil Free Base”; U.S. Patent Publication No. 20050031691 for “Gel Stabilized Nanoparticulate Active Agent Compositions”; U.S. Patent Publication No. 20050019412 for “Novel Glipizide Compositions”; U.S. Patent Publication No. 20050004049 for “Novel Griseofulvin Compositions”; U.S. Patent Publication No. 20040258758 for “Nanoparticulate Topiramate Formulations”; U.S. Patent Publication No. 20040258757 for “Liquid Dosage Compositions of Stable Nanoparticulate Active Agents”; U.S. Patent Publication No. 20040229038 for “Nanoparticulate Meloxicam Formulations”; U.S. Patent Publication No. 20040208833 for “Novel Fluticasone Formulations”; U.S. Patent Publication No. 20040156895 for “Solid Dosage Forms Comprising Pullulan”; U.S. Patent Publication No. 20040156872 for “Novel Nimesulide Compositions”; U.S. Patent Publication No. 20040141925 for “Novel Triamcinolone Compositions”; U.S. Patent Publication No. 20040115134 for “Novel Nifedipine Compositions”; U.S. Patent Publication No. 20040105889 for “Low Viscosity Liquid Dosage Forms”; U.S. Patent Publication No. 20040105778 for “Gamma Irradiation of Solid Nanoparticulate Active Agents”; U.S. Patent Publication No. 20040101566 for “Novel Benzoyl Peroxide Compositions”; U.S. Patent Publication No. 20040057905 for “Nanoparticulate Beclomethasone Dipropionate Compositions”; U.S. Patent Publication No. 20040033267 for “Nanoparticulate Compositions of Angiogenesis Inhibitors”; U.S. Patent Publication No. 20040033202 for “Nanoparticulate Sterol Formulations and Novel Sterol Combinations”; U.S. Patent Publication No. 20040018242 for “Nanoparticulate Nystatin Formulations”; U.S. Patent Publication No. 20040015134 for “Drug Delivery Systems and Methods”; U.S. Patent Publication No. 20030232796 for “Nanoparticulate Polycosanol Formulations & Novel Polycosanol Combinations”; U.S. Patent Publication No. 20030215502 for “Fast Dissolving Dosage Forms Having Reduced Friability”; U.S. Patent Publication No. 20030185869 for “Nanoparticulate Compositions Having Lysozyme as a Surface Stabilizer”; U.S. Patent Publication No. 20030181411 for “Nanoparticulate Compositions of Mitogen-Activated Protein (MAP) Kinase Inhibitors”; U.S. Patent Publication No. 20030137067 for “Compositions Having a Combination of Immediate Release and Controlled Release Characteristics”; U.S. Patent Publication No. 20030108616 for “Nanoparticulate Compositions Comprising Copolymers of Vinyl Pyrrolidone and Vinyl Acetate as Surface Stabilizers”; U.S. Patent Publication No. 20030095928 for “Nanoparticulate Insulin”; U.S. Patent Publication No. 20030087308 for “Method for High Through-put Screening Using a Small Scale Mill or Microfluidics”; U.S. Patent Publication No. 20030023203 for “Drug Delivery Systems & Methods”; U.S. Patent Publication No. 20020179758 for “System and Method for Milling Materials”; and U.S. Patent Publication No. 20010053664 for “Apparatus for Sanitary Wet Milling,” describe nanoparticulate active agent compositions and are specifically incorporated by reference.

Amorphous small particle compositions are described, for example, in U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

B. Background Regarding Fenofibrate

The compositions of the invention comprise a fibrate, preferably fenofibrate. Fenofibrate, also known as 2-[4-(4-chlorobenzoyl)phenoxy]-2-methyl-propanoic acid, 1 methylethyl ester, is a lipid regulating agent. The compound is virtually insoluble in water. See The Physicians' Desk Reference, 56^{th} Ed., pp. 513-516 (2002).

Fenofibrate is described in, for example, U.S. Pat. Nos. 3,907,792 for “Phenoxy-Alkyl-Carboxylic Acid Derivatives and the Preparation Thereof;” 4,895,726 for “Novel Dosage Form of Fenofibrate;” 6,074,670 and 6,277,405, both for “Fenofibrate Pharmaceutical Composition Having High Bioavailability and Method for Preparing It.” U.S. Pat. No. 3,907,792 describes a class of phenoxy-alkyl carboxylic compounds that encompasses fenofibrate. U.S. Pat. No. 4,895,726 describes a gelatin capsule therapeutic composition, useful in the oral treatment of hyerlipidemia and hypercholesterolemia, containing micronized fenofibrate. U.S. Pat. No. 6,074,670 refers to immediate-release fenofibrate compositions comprising micronized fenofibrate and at least one inert hydrosoluble carrier. U.S. Pat. No. 4,739,101 describes a process for making fenofibrate. U.S. Pat. No. 6,277,405 is directed to micronized fenofibrate compositions having a specified dissolution profile. International Publication No. WO 02/24193 for “Stabilized Fibrate Microparticles,” published on Mar. 28, 2002, describes a microparticulate fenofibrate composition comprising a phospholipid. International Publication No. WO 02/067901 for “Fibrate-Statin Combinations with Reduced Fed-Fasted Effects,” published on Sep. 6, 2002, describes a microparticulate fenofibrate composition comprising a phospholipid and a hydroxymethylglutaryl coenzyme A (HMG-CoA) reductase inhibitor or statin. WO 01/80828 for “Improved Water-Insoluble Drug Particle Process,” and International Publication No. WO 02/24193 for “Stabilized Fibrate Microparticles,” describe a process for making small particle compositions of poorly water soluble drugs. The process requires preparing an admixture of a drug and one or more surface-active agents, followed by heating the drug admixture at or above the melting point of the poorly water soluble drug. The heated suspension is then homogenized. The use of such a heating process can be undesirable, as heating a drug to its melting point destroys the crystalline structure of the drug. Upon cooling, a drug may become amorphous or recrystallize in a different isoform, thereby producing a composition, that is physically, and structurally different from that desired. Such a “different” composition may have different pharmacological properties. This is significant as U.S. Food and Drug Administration (USFDA) approval of a drug product requires that the drug substance be stable and produced in a repeatable process.

U.S. Pat. No. 6,368,620 purports to provide two methods for preparing fenofibrate particles of less than 5000 nm or less than 1000 nm in diameter. In a first method, a fibrate is dissolved in a supercritical fluid, the fibrate solution is sprayed through a nozzle to form small particles of the fibrate, resulting small particles of the fibrate are suspended in a liquid in which the particles are insoluble, and the small particles of the fibrate are collected. The second method described in this patent utilized milling to generate a larger quantity of fenofibrate particles. However, the milling process resulted in an undisclosed number of fenofibrate particles having a size of larger than 1 micron, as the patent describes the use of a 1 micron filter to filter the milled composition in an attempt to remove larger sized fenofibrate particles.

WO 03/013474 for “Nanoparticulate Formulations of Fenofibrate,” published on Feb. 20, 2003, describes fibrate compositions comprising vitamin E TPGS (polyethylene glycol (PEG) derivatized vitamin E). The fibrate compositions of this reference comprise particles of fibrate and vitamin E TPGS having a mean diameter from about 100 nm to about 900 nm (page 8, lines 12-15, of WO 03/013474), a D₅₀ of 350-750 nm, and a D₉₉ of 500 to 900 nm (page 9, lines 11-13, of WO 03/013474). The reference does not teach that the described compositions show minimal or no variability when administered in fed as compared to fasted conditions.

A variety of clinical studies have demonstrated that elevated levels of total cholesterol (total-C), low density lipoprotein cholesterol (LDL-C), and apolipoprotein B (apo B), an LDL membrane complex, are associated with human atherosclerosis. Similarly, decreased levels of high density lipoprotein cholesterol (HDL-C) and its transport complex, apolipoprotein A (apo A2 and apo AII), are associated with the development of atherosclerosis. Epidemiologic investigations have established that cardiovascular morbidity and mortality vary directly with the level of total-C, LDL-C, and triglycerides, and inversely with the level of HDL-C. Fenofibric acid, the active metabolite of fenofibrate, produces reductions in total cholesterol, LDL cholesterol, apo-lipoprotein B, total-triglycerides, and triglyceride rich lipoprotein (VLDL) in treated patients. In addition, treatment with fenofibrate results in increases in high density lipoprotein (HDL) and apolipoprotein apo AI and apo AII. See The Physicians' Desk Reference, 56^{th} Ed., pp. 513-516 (2002).

Because fibrates, including fenofibrate, are virtually insoluble in water, achieving acceptable oral bioavailability can be problematic. In addition, conventional fibrate, including fenofibrate, formulations exhibit different biopharmaceutical behavior depending upon the fed or fasted state of the patient. Finally, conventional fibrate, including fenofibrate, formulations require relatively large doses to achieve the desired therapeutic effects. There is a need in the art for nanoparticulate fibrate formulations, that overcome these and other problems associated with prior conventional crystalline fibrate formulations. The present invention satisfies these needs.

### SUMMARY OF THE INVENTION

The present invention relates to nanoparticulate compositions comprising a fibrate, preferably fenofibrate. The compositions comprise a fibrate, preferably fenofibrate, and at least one surface stabilizer adsorbed on the surface of the fibrate particles. The nanoparticulate fibrate, preferably fenofibrate, particles have an effective average particle size of less than about 2000 nm.

A preferred dosage form of the invention is a solid dosage form, although any pharmaceutically acceptable dosage form can be utilized.

Any suitable quantity of a fibrate, such as fenofibrate, can be utilized in the compositions of the invention. Exemplary quantities of a fibrate, such as fenofibrate, comprised in an exemplary dosage form include, but are not limited to, 48 mg, 145 mg, 160 mg, and 200 mg.

Another aspect of the invention is directed to pharmaceutical compositions comprising a nanoparticulate fibrate, preferably fenofibrate, composition of the invention. The pharmaceutical compositions comprise a fibrate, preferably fenofibrate, at least one surface stabilizer, and a pharmaceutically acceptable carrier, as well as any desired excipients. One embodiment of the invention encompasses a fibrate, preferably fenofibrate, composition, wherein the pharmacokinetic profile of the fibrate is not affected by the fed or fasted state of a subject ingesting the composition, in particular as defined by Cₘₐₓ and AUC guidelines established by the U.S. Food and Drug Administration and the corresponding European regulatory agency (EMEA).

Another aspect of the invention is directed to a nanoparticulate fibrate, preferably fenofibrate, composition having improved pharmacokinetic profiles as compared to conventional microcrystalline fibrate formulations, such as Tₘₐₓ, Cₘₐₓ, and AUC. In a different embodiment, the invention encompasses a fibrate, preferably fenofibrate, composition, wherein administration of the composition to a subject in a fasted state is bioequivalent to administration of the composition to a subject in a fed state, in particular as defined by Cₘₐₓ and AUC guidelines established by the U.S. Food and Drug Administration and the corresponding European regulatory agency (EMEA).

Another embodiment of the invention is directed to nanoparticulate fibrate, preferably fenofibrate, compositions additionally comprising one or more compounds useful in treating dyslipidemia, hyperlipidemia, hypercholesterolemia, cardiovascular disorders, or related conditions.

In one embodiment of the invention, the fibrate compositions of the invention comprise sucrose. In another embodiment of the invention, the fibrate compositions of the invention do not contain any trace residues of a supercritical fluid from the manufacturing process used to make the compositions. In yet another embodiment, the fibrate compositions of the invention do not require filtering, following milling, to obtain a composition in which the fibrate has a D50 particle size of less than 1 micron and/or a D90 of less than about 2 microns. In yet another embodiment, the fibrate compositions of the invention have a narrow particle size distribution curve. This means that the size of the fibrate particles within the composition does not vary significantly. A narrow particle size distribution is preferred, as widely variable particle sizes could potentially result in inconsistent bioavailability from dose to dose, as larger fibrate particles will dissolve much slower than smaller fibrate particles following administration.

Other embodiments of the invention include, but are not limited to, nanoparticulate fibrate, preferably fenofibrate, formulations which, as compared to conventional non-nanoparticulate formulations of a fibrate, particularly a fenofibrate such as TRICOR® (160 mg tablet or 200 mg capsule microcrystalline fenofibrate formulations), have one or more of the following properties: (1) smaller tablet or other solid dosage form size; (2) smaller doses of drug required to obtain the same pharmacological effect (3) increased bioavailability; (4) substantially similar pharmacokinetic profiles of the nanoparticulate fibrate, preferably fenofibrate, compositions when administered in the fed versus the fasted state; and (5) an increased rate of dissolution for the nanoparticulate fibrate, preferably fenofibrate, compositions.

This invention further discloses a method of making a nanoparticulate fibrate, preferably fenofibrate, composition according to the invention. Such a method comprises contacting a fibrate, preferably fenofibrate, and at least one surface stabilizer for a time and under conditions sufficient to provide a nanoparticulate fibrate composition, and preferably a fenofibrate composition. The one or more surface stabilizers can be contacted with a fibrate, preferably fenofibrate, either before, during, or after size reduction of the fibrate. The present invention is also directed to methods of treatment using the nanoparticulate fibrate, preferably fenofibrate, compositions of the invention for conditions such as hypercholesterolemia, hypertriglyceridemia, coronary heart disease, and peripheral vascular disease (including symptomatic carotid artery disease). The compositions of the invention can be used as adjunctive therapy to diet for the reduction of LDL-C, total-C, triglycerides, and Apo B in adult patients with primary hypercholesterolemia or mixed dyslipidemia (Fredrickson Types IIa and IIb). The compositions can also be used as adjunctive therapy to diet for treatment of adult patients with hypertriglyceridemia (Fredrickson Types IV and V hyperlipidemia).

Markedly elevated levels of serum tryglycerides (e.g., >2000 mg/dL) may increase the risk of developing pancreatitis. Such methods comprise administering to a subject a therapeutically effective amount of a nanoparticulate fibrate, preferably fenofibrate, composition according to the invention. Other methods of treatment using the nanoparticulate compositions of the invention are known to those skilled in the art.

Both the foregoing general description, and the following detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1: Shows the fenofibric acid concentration (μg/ml) over a period of 120 hours for a single dose of: (a) a 160 mg nanoparticulate fenofibrate tablet administered to a fasting subject; (b) a 160 mg nanoparticulate fenofibrate tablet administered to a high fat fed subject; and (c) a 200 mg microcrystalline (TRICOR®; Abbott Laboratories, Abbott Park, Ill.) capsule administered to a low fat fed subject; and

FIG. 2: Shows the fenofibric acid concentration (μg/ml) over a period of 24 hours for a single dose of: (a) a 160 mg nanoparticulate fenofibrate tablet administered to a fasting subject; (b) a 160 mg nanoparticulate fenofibrate tablet administered to a high fat fed subject; and (c) a 200 mg microcrystalline (TRICOR®) capsule administered to a low fat fed subject.

### DETAILED DESCRIPTION OF THE INVENTION

The present invention is directed to nanoparticulate compositions comprising a fibrate, preferably fenofibrate. The compositions comprise a fibrate, preferably fenofibrate, and preferably at least one surface stabilizer adsorbed on the surface of the drug. The nanoparticulate fibrate, preferably fenofibrate, particles have an effective average particle size of less than about 2000 nm.

As taught in the '684 patent, and as exemplified in the examples below, not every combination of surface stabilizer and active agent will result in a stable nanoparticulate composition. It was surprisingly discovered that stable, nanoparticulate fibrate, preferably fenofibrate, formulations can be made.

Advantages of the nanoparticulate fibrate, preferably fenofibrate, formulations of the invention as compared to conventional non-nanoparticulate formulations of a fibrate, particularly a fenofibrate such as TRICOR® (tablet or capsule microcrystalline fenofibrate formulations), include, but are not limited to: (1) smaller tablet or other solid dosage form size; (2) smaller doses of drug required to obtain the same pharmacological effect; (3) increased bioavailability; (4) substantially similar pharmacokinetic profiles of the nanoparticulate fibrate, preferably fenofibrate, compositions when administered in the fed versus the fasted state; (5) improved pharmacokinetic profiles; (6) bioequivalency of the nanoparticulate fibrate, preferably fenofibrate, compositions when administered in the fed versus the fasted state; and (7) an increased rate of dissolution for the nanoparticulate fibrate, preferably fenofibrate, compositions.

The present invention also includes nanoparticulate fibrate, preferably fenofibrate, compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, otic, intracisternal, intraperitoneal, or topical administration, and the like. A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

In one embodiment of the invention, the fibrate dosage form comprises sucrose as an excipient and DOSS and hypromellose as surface stabilizers.

The present invention is described herein using several definitions, as set forth below and throughout the application. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to stable fibrate, preferably fenofibrate, particles, “stable” includes, but is not limited to, one or more of the following parameters: (1) that the fibrate particles do not appreciably flocculate or agglomerate due to interparticle attractive forces, or otherwise significantly increase in particle size over time; (2) that the physical structure of the fibrate, preferably fenofibrate, particles is not altered over time, such as by conversion from an amorphous phase to crystalline phase; (3) that the fibrate, preferably fenofibrate, particles are chemically stable; and/or (4) where the fibrate has not been subject to a heating step at or above the melting point of the fibrate in the preparation of the nanoparticles of the invention.

A. Preferred Characteristics of the Fibrate Compositions of the Invention

1. Increased Bioavailability

The fibrate, preferably fenofibrate, formulations of the present invention exhibit increased bioavailability relative to conventional fibrate, preferably fenofibrate, formulations, and therefore require administration of smaller doses of the drug to achieve equivalent pharmacokinetic profiles. Under U.S. FDA guidelines, two products (or treatments) may be deemed bioequivalent if the 90% confidence intervals (CI) for AUC and Cₘₐₓ fall between 80% and 125%. According to Europe's EMEA guidelines, the 90% CI for AUC must fall between 80% and 125%, and the 90% CI for Cₘₐₓ must fall between 70% and 143%.

As shown below in Example 6, administration of a 160 mg nanoparticulate fenofibrate tablet in the fed state is found to be bioequivalent to administration of a 200 mg conventional microcrystalline fenofibrate capsule (TRICOR®) in the fed state. Thus, the nanoparticulate fenofibrate dosage form requires less drug (160 mg vs. 200 mg) to achieve a pharmacokinetic profile that is equivalent, based upon AUC and Cₘₐₓ, to the conventional microcrystalline fenofibrate dosage form (e.g., TRICOR®). Therefore, the nanoparticulate fenofibrate dosage form exhibits increased bioavailability relative to the conventional microcrystalline fenofibrate dosage form (e.g., TRICOR®).

Greater bioavailability of the fibrate compositions of the invention can enable a smaller solid dosage size. This is particularly significant for patient populations such as the elderly, juvenile, and infant. In one embodiment of the invention, disclosed is a stable solid dose fenofibrate composition comprising: (a) a therapeutically effective dosage of 145 mg of particles of fenofibrate or a salt thereof; and (b) associated with the surface thereof at least one surface stabilizer. Characteristics of the composition include: (i) the fenofibrate particles have an effective average particle size of less than about 2000 nm; (ii) the dosage form exhibits an increased AUC as compared to the microcrystalline fenofibrate 200 mg tablet; (iii) the dosage form exhibits an increased Cₘₐₓ as compared to the microcrystalline fenofibrate 200 mg tablet; (iv) the dosage form exhibits an increased Cₘₐₓ and an increased AUC as compared to the microcrystalline fenofibrate 200 mg; (v) the solid dose is bioequivalent to the microcrystalline fenofibrate 200 mg tablet, wherein bioequivalency is established by a 90% Confidence Interval of between 80% and 125% for both Cₘₐₓ and AUC; (vi) the solid dose is bioequivalent to the microcrystalline fenofibrate 200 mg tablet, wherein bioequivalency is established by a 90% Confidence Interval of between 80% and 125% for AUC and a 90% Confidence Interval of between 70% and 143% for Cₘₐₓ; and/or (vii) the solid dose is about 10% smaller than the microcrystalline TRICOR® 160 mg tablet.

In another embodiment of the invention, disclosed is a stable solid dose fenofibrate composition comprising: (a) a therapeutically effective dosage of 48 mg of particles of fenofibrate or a salt thereof; and (b) associated with the surface thereof at least one surface stabilizer. Characteristics of the composition include: (i) the fenofibrate particles have an effective average particle size of less than about 2000 nm; (ii) the dosage form exhibits an increased AUC as compared to the microcrystalline fenofibrate 67 mg; (iii) the dosage form exhibits an increased Cₘₐₓ as compared to the microcrystalline fenofibrate 67 mgtablet; (iv) the dosage form exhibits an increased Cₘₐₓ and an increased AUC as compared to the microcrystalline fenofibrate 67 mgtablet; (v) the solid dose is bioequivalent to the microcrystalline fenofibrate 67 mgtablet, wherein bioequivalency is established by a 90% Confidence Interval of between 80% and 125% for both Cₘₐₓ and AUC; (vi) the solid dose is bioequivalent to the microcrystalline fenofibrate 67 mgtablet, wherein bioequivalency is established by a 90% Confidence Interval of between 80% and 125% for AUC and a 90% Confidence Interval of between 70% and 143% for Cₘₐₓ; and (vii) the solid dose is about 10% smaller than the microcrystalline fenofibrate 67 mgtablet.

2. Improved Pharmacokinetic Profiles

The invention also provides fibrate, preferably fenofibrate, compositions having a desirable pharmacokinetic profile when administered to mammalian subjects. The desirable pharmacokinetic profile of the fibrate, preferably fenofibrate, compositions comprise the parameters: (1) that the Tₘₐₓ of a fibrate, preferably fenofibrate, when assayed in the plasma of the mammalian subject, is less than about 6 to about 8 hours. Preferably, the Tₘₐₓ parameter of the pharmacokinetic profile is less than about 6 hours, less than about 5 hours, less than about 4 hours, less than about 3 hours, less than about 2 hours, less than about 1 hour, or less than about 30 minutes after administration. The desirable pharmacokinetic profile, as used herein, is the pharmacokinetic profile measured after the initial dose of a fibrate, preferably fenofibrate. The compositions can be formulated in any way as described below and as known to those skilled in the art.

Current marketed formulations of fenofibrate include tablets, i.e., microcrystalline TRICOR® tablets marketed by Abbott Laboratories. According to the description of TRICOR®, the pharmacokinetic profile of the tablets contain parameters such that the median Tₘₐₓ is 6-8 hours (Physicians Desk Reference, 56^{th} Ed., 2002). Because the compound is virtually insoluble in water, the absolute bioavailability of microcrystalline TRICOR® cannot be determined (Physicians Desk Reference, 56^{th} Ed., 2002). The compositions of the invention improve upon at least the Tₘₐₓ parameter of the pharmacokinetic profile of a fibrate, preferably fenofibrate.

A preferred fibrate formulation, preferably a fenofibrate formulation, of the invention exhibits in comparative pharmacokinetic testing with a standard commercial formulation of the same fibrate, e.g., microcrystalline TRICOR® tablets from Abbott Laboratories for fenofibrate, a Tₘₐₓ not greater than about 90%, not greater than about 80%, not greater than about 70%, not greater than about 60%, not greater than about 50%, not greater than about 30%, or not greater than about 25% of the Tₘₐₓ exhibited by a standard commercial fibrate formulation, e.g., microcrystalline TRICOR® tablets for fenofibrate.

Any formulation giving the desired pharmacokinetic profile is suitable for administration according to the present methods. Exemplary types of formulations giving such profiles are liquid dispersions, gels, aerosols, ointments, creams, solid dose forms, etc. of a nanoparticulate fibrate, preferably nanoparticulate fenofibrate.

In a preferred embodiment of the invention, a fenofibrate composition of the invention comprises fenofibrate or a salt thereof, which when administered to a human as a dose of about 160 mg presents an AUC of about 139 μg/mL·h.

3. The Pharmacokinetic Profiles of the Fibrate Compositions of the Invention are not Affected by the Fed or Fasted State of the Subject Ingesting the Compositions

The invention encompasses a fibrate, preferably fenofibrate, composition wherein the pharmacokinetic profile of the fibrate is not substantially affected by the fed or fasted state of a subject ingesting the composition, when administered to a human. This means that there is no substantial difference in the quantity of drug absorbed (as measured by AUC) or the rate of drug absorption (as measured by Cₘₐₓ) when the nanoparticulate fibrate, preferably fenofibrate, compositions are administered in the fed versus the fasted state.

In one embodiment of the invention, “fasting conditions” and “fed conditions” are as defined by the U.S. Food and Drug Administration. According to U.S. Food and Drug Administration guidelines, “fasted conditions” consist of an overnight fast of at least 10 hours prior to administration of the composition to be tested. Generally, subjects should be administered the drug product with 240 mL (8 fluid ounces) of water. Preferably, no food should be allowed for at least 4 hours post-dose. Water can be allowed as desired except for one hour before and after drug administration. See U.S. Department of Health and Human Services, Food and Drug Administration, Center for Drug Evaluation and Research (CDER), December 2002, BP, Guidance for Industry, “Food-Effect Bioavailability and Fed Bioequivalence Studies.”

Also according to the U.S. FDA, “fed conditions” consist of an overnight fast of at least 10 hours, following by ingestion of a recommended meal 30 minutes prior to administration of the drug product. Study subjects should eat this meal in 30 minutes or less; however, the drug product should be administered 30 minutes after start of the meal. The drug product should be administered with 240 mL (8 fluid ounces) of water. No food should be allowed for at least 4 hours post-dose. Water can be allowed as desired except for one hour before and after drug administration. Id.

The U.S. FDA recommends that studies be conducted using meal conditions that are expected to provide the greatest effects on GI physiology so that systemic drug availability is maximally affected. A high-fat (approximately 50 percent of total caloric content of the meal) and high-calorie (approximately 800 to 1000 calories) meal is recommended as a test meal for food-effect bioavailability and fed bioequivalency studies. This test meal should derive approximately 150, 250, and 500-600 calories from protein, carbohydrate, and fat, respectively.

As described in the examples below, “fasting” conditions are defined as no food or beverage, except for water to quench thirst, beginning 10 hours before dosing. “low fat fed” conditions are defined as 30% fat-400 Kcal, and “high fat fed” conditions are defined as 50% fat-1000 Kcal.

For conventional fenofibrate formulations, i.e., microcrystalline TRICOR®, the absorption of fenofibrate is increased by approximately 35% when administered with food. This significant difference in absorption observed with conventional fenofibrate formulations is undesirable. The fibrate, preferably fenofibrate, formulations of the invention overcome this problem, as the fibrate formulations reduce or preferably substantially eliminate significantly different absorption levels when administered under fed as compared to fasting conditions when administered to a human. In one embodiment of the invention, the fibrate, preferably fenofibrate, dosage form exhibits no substantial difference between the AUC of the composition when administered to a human subject under fed versus fasted conditions. In another embodiment, the fibrate, preferably fenofibrate, dosage form exhibits no substantial difference between the Cₘₐₓ of the composition when administered to a human subject under fed versus fasted conditions. In yet another embodiment, the fibrate, preferably fenofibrate, dosage form exhibits no substantial difference between the AUC, and no substantial difference between the Cₘₐₓ, of the composition when administered to a human subject under fed versus fasted conditions. In one embodiment of the invention, a fenofibrate composition of the invention comprises about 145 mg of fenofibrate and exhibits minimal or no food effect when administered to a human. Preferably, the 145 mg fenofibrate dosage form: (i) exhibits no substantial difference between the AUC of the composition when administered to a human subject under fed versus fasted conditions; (ii) exhibits no substantial difference between the Cₘₐₓ of the composition when administered to a human subject under fed versus fasted conditions; or (iii) exhibits no substantial difference between the AUC, and no substantial difference between the Cₘₐₓ, of the composition when administered to a human subject under fed versus fasted conditions.

In another preferred embodiment of the invention, a fenofibrate composition of the invention comprises about 48 mg of fenofibrate and exhibits minimal or no food effect when administered to a human. Preferably, the 48 mg fenofibrate dosage form: (i) exhibits no substantial difference between the AUC of the composition when administered to a human subject under fed versus fasted conditions; (ii) exhibits no substantial difference between the Cₘₐₓ of the composition when administered to a human subject under fed versus fasted conditions; or (iii) exhibits no substantial difference between the AUC, and no substantial difference between the Cₘₐₓ, of the composition when administered to a human subject under fed versus fasted conditions.

In another embodiment of the invention, the fenofibrate compositions exhibit an AUC which does not substantially differ, when administered under fed as compared to fasting conditions. In other embodiments of the invention, the AUC can differ by about 40% or less, about 35% or less, about 30% or less, about 25% or less, about 20% or less, about 15% or less, about 10% or less, about 5% or less, or about 3% or less, when administered under fed as compared to under fasting conditions. Exemplary fenofibrate compositions include, but are not limited to, fenofibrate compositions comprising about 145 mg of fenofibrate or about 48 mg of fenofibrate. In another embodiment of the invention, the fenofibrate compositions exhibit a Cₘₐₓ which does not substantially differ, when administered under fed as compared to fasting conditions. In other embodiments of the invention, the Cₘₐₓ can differ by about 60% or less, about 55% or less, about 50% or less, about 45% or less, about 40% or less, about 35% or less, about 30% or less, about 25% or less, about 20% or less, about 15% or less, about 10% or less, about 5% or less, or about 3% or less, when administered under fed as compared to under fasting conditions. Exemplary fenofibrate compositions include, but are not limited to, fenofibrate compositions comprising about 145 mg of fenofibrate or about 48 mg of fenofibrate.

As shown in Example 6, the pharmacokinetic parameters of the fenofibrate compositions of the invention are the same when the composition is administered in the fed and fasted states when administered to a human. Specifically, there was no substantial difference in the rate or quantity of drug absorption when the fenofibrate composition was administered in the fed versus the fasted state. Thus, the fibrate compositions, and preferably fenofibrate compositions, of the invention substantially eliminate the effect of food on the pharmacokinetics of the fibrate when administered to a human. Benefits of a dosage form which substantially eliminates the effect of food include an increase in subject convenience, thereby increasing subject compliance, as the subject does not need to ensure that they are taking a dose either with or without food. This is significant, as with poor subject compliance a worsening of the medical condition for which the drug is being prescribed may be observed.

4. Bioequivalency of the Fibrate Compositions of the Invention when Administered in the Fed Versus the Fasted State

The invention also encompasses a fibrate, preferably a fenofibrate, composition in which administration of the composition to a subject in a fasted state is bioequivalent to administration of the composition to a subject in a fed state. “Bioequivalency” under U.S. FDA regulatory guidelines can be established by a 90% Confidence Interval (CI) of between 80% and 125% for both Cₘₐₓ and AUC. Under the European EMEA regulatory guidelines, “bioequivalency” is established with a 90% CI for AUC of between 80% and 125%, and a 90% CI for Cₘₐₓ of between 70% and 143%. The difference in absorption of the fibrate, preferably fenofibrate, compositions of the invention, when administered in the fed versus the fasted state, preferably is less than about 35%, less than about 30%, less than about 25%, less than about 20%, less than about 15%, less than about 10%, less than about 5%, or less than about 3%.

As shown in Example 6, administration of a fenofibrate composition according to the invention in a fasted state was bioequivalent to administration of a fenofibrate composition according to the invention in a fed state, pursuant to regulatory guidelines. Under USFDA guidelines, two products or treatments are bioequivalent if the 90% Confidence Intervals (CI) for Cₘₐₓ (peak concentration) and the AUC (area under the concentration/time curve) are between 80% and 125%. For Europe, the criterion for bioequivalency of two products or treatments is a 90% CI for AUC of between 80% and 125%, and a 90% CI for Cₘₐₓ of between 70% and 143% realtive to the reference listed product. The fibrate, preferably fenofibrate, compositions of the invention meet both the U.S. and European guidelines for bioequivalency for administration in the fed versus the fasted state.

Prior to the present invention, formulations of fenofibrate failed to exhibit bioequivalency under fed and fasting conditions, per U.S. FDA guidelines. In particular, prior researchers found it particularly difficult to meet the Cₘₐₓ parameters of the U.S. FDA' guidelines to establish bioequivalency under fed and fasting conditions. For example, U.S. Pat. No. 6,696,084 to Pace et al. (“Pace”) purportedly teaches a fenofibrate composition that “substantially reduces or substantially eliminates the difference in the amount of the drug or active fibrate species taken up in the patient when in a fasting state versus the amount taken up using the same dosage level in the same patient when in a fed state.” Pace at col. 16, lines 56-65. However, a “reduction” in fed/fasted variability is not the same as achieving “bioequivalency” between the fed/fasted pharmacokinetic profiles, as provided by one embodiment of the present invention. Moreover, Pace was unsuccessful in obtaining their “object” of reducing fed/fasted variability. Utilizing the formulation identified by Pace, subsequent work by the Pace researchers showed they were able to reduce the fed/fasted variability for AUC, but they failed to reduce fed/fasted variability for Cₘₐₓ. Specifically, Pace describes the preparation of fenofibrate formulations with various phospholipids as the surface active substance, including Lipoid E80, Phospholipon 100H, and Phospholipon 90H. See e.g., Tables 2 and 3, col. 32, of Pace. No in vivo data is described in Pace for the disclosed fenofibrate compositions. However, a related application continues the research described in Pace by utilizing the Pace compositions Pace in vivo. The results of this in vivo testing showed that Pace's fenofibrate formulations dramatically failed to reduce fed/fasted variability for Cₘₐₓ. US 2003/0194442 (“the '442 application”) is related to Pace: the '442 application and Pace both claim priority to U.S. Provisional Application Nos. 60/234,186, filed on Sep. 20, 2000, and 60/241,761, filed on Oct. 20, 2000. The '442 application provides in vivo data for Pace's fenofibrate compositions, and teaches that Pace's fenofibrate compositions fail to meet the Cₘₐₓ limitation of the U.S. FDA's requirement to establish bioequivalency under fed and fasting conditions. Specifically, Example 19 of the '442 application describes in vivo oral bioavailability of a fenofibrate composition having Phospholipon 100H as the surface active substance (i.e., the same composition described in Pace) under fed and fasted conditions. See page 28 of the '442 application. The example reports a 13% difference in AUC, measured under fed and fasted conditions (see Example 19 and Table 6). However, the '442 application also reports that the Cₘₐₓ, when measured under fed and fasted conditions, differed by 61%. See Page 7, paragraph 58, of the '442 application. A 61% difference between the Cₘₐₓ measured under fed and fasted conditions does not meet the U.S. FDA's requirement of a 90% CI for Cₘₐₓ of between 80% to 125% to establish bioequivalency between fed and fasting conditions.

5. Dissolution Profiles of the Fibrate Compositions of the Invention

The fibrate, preferably fenofibrate, compositions of the invention have unexpectedly rapid dissolution profiles. Rapid dissolution of an administered active agent is preferable, as faster dissolution may lead to faster onset of action and greater bioavailability. The fibrate, preferably fenofibrate, compositions of the invention preferably have a dissolution profile in which within about 5 minutes at least about 20% of the composition is dissolved. In other embodiments of the invention, at least about 30% or about 40% of the fibrate, preferably fenofibrate, composition is dissolved within about 5 minutes. In yet other embodiments of the invention, preferably at least about 40%, about 50%, about 60%, about 70%, or about 80% of the fibrate, preferably fenofibrate, composition is dissolved within about 10 minutes. Finally, in another embodiment of the invention, preferably at least about 70%, about 80%, about 90%, or about 100% of the fibrate, preferably fenofibrate, composition is dissolved within about 20 minutes. Dissolution is preferably measured in a medium which is discriminating. Such a dissolution medium is intended to produce different in vitro dissolution profiles for two products having different in vivo dissolution profiles in gastric juices; i.e., the dissolution behavior of the products in the dissolution medium is intended to be predictive of the dissolution behavior within the body. An exemplary dissolution medium is an aqueous medium containing the surfactant sodium lauryl sulfate at a concentration of 0.025 M. Determination of the amount dissolved can be carried out by spectrophotometry. The rotating blade method (European Pharmacopoeia) can be used to measure dissolution.

6. Redispersibility Profiles of the Fibrate Compositions of the Invention

An additional feature of the fibrate, preferably fenofibrate, compositions of the invention is that the compositions redisperse such that the effective average particle size of the redispersed fibrate particles is less than about 2 microns. This is significant, as if upon administration the nanoparticulate fibrate compositions of the invention did not redisperse to a substantially nanoparticulate particle size, then the dosage form might lose the benefits afforded by formulating the fibrate into a nanoparticulate particle size. This is because nanoparticulate active agent compositions benefit from the small particle size of the active agent; if the active agent does not redisperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall well below that observed when the nanoparticulate active agent is well dispersed.

Moreover, the nanoparticulate fibrate, preferably fenofibrate, compositions of the invention are believed to exhibit extensive redispersibility of the nanoparticulate fibrate particles upon administration to a mammal, such as a human or animal, as demonstrated by reconstitution/redispersibility in a biorelevant aqueous medium such that the effective average particle size of the redispersed fibrate particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found within the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14M. See e.g., Lindahl et al., “Characterization of Fluids from the Stomach and Proximal Jejunum in Men and Women,” Pharm. Res., 14 (4): 497-502 (1997). It is believed that the pH and ionic strength of the test solution are more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc. Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed fibrate, preferably fenofibrate, particles of the invention (redispersed in an aqueous, biorelevant, or any other suitable medium) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 11000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

7. Fibrate Compositions Used in Conjunction with Other Active Agents

The fibrate, preferably fenofibrate, compositions of the invention can additionally comprise one or more compounds useful in treating dyslipidemia, hyperlipidemia, hypercholesterolemia, cardiovascular disorders, or related conditions, or the fibrate, preferably fenofibrate, compositions can be administered in conjunction with such a compound. Other examples of such compounds include, but are not limited to, CETP (cholesteryl ester transfer protein) inhibitors (e.g., torcetrapib), cholesterol lowering compounds (e.g., ezetimibe (Zetia®)) antihyperglycemia agents, statins or HMG CoA reductase inhibitors and antihypertensives. Examples of antihypertensives include, but are not limited to diuretics (“water pills”), beta blockers, alpha blockers, alpha-beta blockers, sympathetic nerve inhibitors, angiotensin converting enzyme (ACE) inhibitors, calcium channel blockers, angiotensin receptor blockers (formal medical name angiotensin-2-receptor antagonists, known as “sartans” for short). Examples drugs useful in treating hyperglycemia include, but are not limited to, (a) insulin (Humulin®, Novolin®), (b) sulfonylureas, such as glyburide (Diabeta®, Micronase®), acetohexamide (Dymelor®), chlorpropamide (Diabinese®), glimepiride (Amaryl®), glipizide (Glucotrol®), gliclazide, tolazamide (Tolinase®), and tolbutamide (Orinase®), (c) meglitinides, such as repaglinide (Prandin®) and nateglinide (Starlix®), (d) biguanides such as metformin (Glucophage®, Glycon,), (e) thiazolidinediones such as rosiglitazone (Avandia®) and pioglitazone (Actos®), and (f) glucosidase inhibitors, such as acarbose (Precose®) and miglitol (Glyset®).

Examples of statins or HMG CoA reductase inhibitors include, but are not limited to, lovastatin (Mevacor®, Altocor®); pravastatin (Pravachol®); simvastatin (Zocor®); velostatin; atorvastatin (Lipitor®) and other 6-[2-(substituted-pyrrol-1-yl)alkyl]pyran-2 ones and derivatives, as disclosed in U.S. Pat. No. 4,647,576); fluvastatin (Lescol®); fluindostatin (Sandoz XU-62-320); pyrazole analogs of mevalonolactone derivatives, as disclosed in PCT application WO 86/03488; rivastatin (also known as cerivastatin, Baycol®) and other pyridyldihydroxyheptenoic acids, as disclosed in European Patent 491226A; Searle's SC-45355 (a 3-substituted pentanedioic acid derivative); dichloroacetate; imidazole analogs of mevalonolactone, as disclosed in PCT application WO 86/07054; 3-carboxy-2-hydroxy-propane-phosphonic acid derivatives, as disclosed in French Patent No. 2,596,393; 2,3-di-substituted pyrrole, furan, and thiophene derivatives, as disclosed in European Patent Application No. 0221025; naphthyl analogs of mevalonolactone, as disclosed in U.S. Pat. No. 4,686,237; octahydronaphthalenes, such as those disclosed in U.S. Pat. No. 4,499,289; keto analogs of mevinolin (lovastatin), as disclosed in European Patent Application No. 0,142,146 A2; phosphinic acid compounds; rosuvastatin (Crestor®); pitavastatin (Pitava®), as well as other HMG CoA reductase inhibitors.

B. Compositions

The invention provides compositions comprising fibrate, preferably fenofibrate, particles, and at least one surface stabilizer. The surface stabilizers preferably are adsorbed on, or associated with, the surface of the fibrate, preferably fenofibrate, particles. Surface stabilizers especially useful herein preferably physically adhere on, or associate with, the surface of the nanoparticulate fibrate particles but do not chemically react with the fibrate particles or itself. Individually adsorbed molecules of the surface stabilizer are essentially free of intermolecular cross-linkages.

The present invention also includes fibrate, preferably fenofibrate, compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, otic, local (powders, ointments or drops), buccal, intracisternal, intraperitoneal, or topical administration, and the like.

The fibrate compositions can be formulated for administration via any suitable method, such as parenteral injection (e.g., intravenous, intramuscular, or subcutaneous), oral administration (in solid, liquid, or aerosol (i.e., pulmonary) form), vaginal, nasal, rectal, ocular, otic, local (powders, creams, ointments or drops), buccal, intracisternal, intraperitoneal, topical administration, and the like. Exemplary fibrate dosage forms of the invention include, but are not limited to, liquid dispersions, gels, powders, sprays, solid re-dispersible dosage forms, ointments, creams, aerosols (pulmonary and nasal), solid dose forms, etc. In other embodiments of the invention, the fibrate compositions can be formulated: (a) for administration selected from the group consisting of parenteral, oral, pulmonary, intravenous, rectal, ophthalmic, colonic, intracisternal, intravaginal, intraperitoneal, ocular, otic, local, buccal, nasal, bioadhesive and topical administration; (b) into a dosage form selected from the group consisting of liquid dispersions, gels, aerosols, ointments, creams, lyophilized formulations, tablets, capsules; (c) into a dosage form selected from the group consisting of controlled release formulations, fast melt formulations, delayed release formulations, extended release formulations, pulsatile release formulations, mixed immediate release formulations, controlled release formulations; or (d) any combination of (a), (b), and (c).

1. Fibrate Particles

As used herein the term “fibrate” means any of the fibric acid derivatives useful in the methods described herein, e.g., fenofibrate. Fenofibrate is a fibrate compound, other examples of which are bezafibrate, beclobrate, binifibrate, ciplofibrate, clinofibrate, clofibrate, clofibric acid, etofibrate, gemfibrozil, nicofibrate, pirifibrate, ronifibrate, simfibrate, theofibrate, etc. See U.S. Pat. No. 6,384,062. Generally, fibrates are used for conditions such as hypercholesterolemia, mixed lipidemia, hypertriglyceridemia, coronary heart disease, and peripheral vascular disease (including symptomatic carotid artery disease), and prevention of pancreatitis. Fenofibrate may also help prevent the development of pancreatitis (inflammation of the pancreas) caused by high levels of triglycerides in the blood. Fibrates are known to be useful in treating renal failure (U.S. Pat. No. 4,250,191). Fibrates may also be used for other indications where lipid regulating agents are typically used. As used herein the term “fenofibrate” is used to mean fenofibrate (2-[4-(4 chlorobenzoyl)phenoxy]-2-methyl-propanoic acid, 1-methylethyl ester) or a salt thereof. Fenofibrate is well known in the art and is readily recognized by one of ordinary skill. It is used to lower triglyceride (fat-like substances) levels in the blood. Specifically, fenofibrate reduces elevated LDL-C, Total-C, triglycerides, and Apo-B and increases HDL-C. The drug has also been approved as adjunctive therapy for the treatment of hypertriglyceridemia, a disorder characterized by elevated levels of very low density lipoprotein (VLDL) in the plasma. The mechanism of action of fenofibrate has not been clearly established in man. Fenofibric acid, the active metabolite of fenofibrate, lowers plasma triglycerides apparently by inhibiting triglyceride synthesis, resulting in a reduction of VLDL released into the circulation, and also by stimulating the catabolism of triglyceride-rich lipoprotein (i.e., VLDL). Fenofibrate also reduces serum uric acid levels in hyperuricemic and normal individuals by increasing the urinary excretion of uric acid. The absolute bioavailability of conventional microcrystalline fenofibrate cannot be determined as the compound is virtually insoluble in aqueous media suitable for injection.

However, fenofibrate is well absorbed from the gastrointestinal tract. Following oral administration in healthy volunteers, approximately 60% of a single dose of conventional radiolabelled fenofibrate (i.e., microcrystalline TRICOR®) appeared in urine, primarily as fenofibric acid and its glucuronate conjugate, and 25% was excreted in the feces. See http://www.rxlist.com/cgi/generic3/fenofibrate_cp.htm

Following oral administration, fenofibrate is rapidly hydrolyzed by esterases to the active metabolite, fenofibric acid; no unchanged fenofibrate is detected in plasma. Fenofibric acid is primarily conjugated with glucuronic acid and then excreted in urine. A small amount of fenofibric acid is reduced at the carbonyl moiety to a benzhydrol metabolite which is, in turn, conjugated with glucuronic acid and excreted in urine. Id.

Any suitable quantity of a fibrate, such as fenofibrate, can be utilized in the compositions of the invention. Exemplary quantities of a fibrate, such as fenofibrate, comprised in an exemplary dosage form include, but are not limited to, 48 mg, 145 mg, 160 mg, and 200 mg. Other exemplary quantities of a fibrate, such as fenofibrate, that can be included in the compositions of the invention include, but are not limited to, any amount between 10 mg and 500 mg, in single mg increments (e.g., 10 mg, 11 mg, 12 mg, . . . 498 mg, 499 mg, or 500 mg).

2. Surface Stabilizers

The choice of a surface stabilizer for a fibrate is non-trivial and required extensive experimentation to realize a desirable formulation. Accordingly, the present invention is directed to the surprising discovery that nanoparticulate fibrate, preferably fenofibrate, compositions can be made. Combinations of more than one surface stabilizer can be used in the invention.

Useful surface stabilizers, which can be employed in the invention, include, but are not limited to, known organic and inorganic pharmaceutical excipients. Such excipients include various polymers, low molecular weight oligomers, natural products, and surfactants. Surface stabilizers include nonionic, anionic, cationic, ionic, and zwitterionic surfactants and compounds.

Representative examples of surface stabilizers useful in the invention include, but are not limited to, albumin, including but not limited to human serum albumin and bovine albumin, hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA9OHCO, which is C18H37CH2(CON(CH3)-CH2(CHOH)4(CH20H)2 (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptylβ-D-glucopyranoside; n-heptyl β-D-thioglucoside; n-hexyl β-D-glucopyranoside; nonanoyl-N-methylglucamide; n-noyl β-D-glucopyranoside; octanoyl-Nmethylglucamide; n-octyl-β-D-glucopyranoside; octyl β-D-thioglucopyranoside; Albumin; Bovine Serum Albumin (BSA); Human Serum Albumin (HAS); Delipidated Albumin, either HSA or BSA; Factor V Albumin; HSAPEG-phospholipid, PEG-cholesterol, PEG-cholesterol derivative, PEG-vitamin A, PEG-vitamin E, lysozyme, random copolymers of vinyl pyrrolidone and vinyl acetate, and the like. If desirable, the nanoparticulate fibrate, preferable fenofibrate, compositions of the invention can be formulated to be phospholipid-free.

Examples of useful cationic surface stabilizers include, but are not limited to, olymers, biopolymers, polysaccharides, cellulosics, alginates, phospholipids, and onpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethylammonium chloride or bromide, decyl triethyl ammonium chloride, decyl dimethylhydroxyethyl ammonium chloride or bromide, C₁₂₋₁₅-dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyldimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, d) 1-cysteine, N tetradecyldimethylbenzyl ammonium, chloride monohydrate, N-alkyl(C₁₂₋₁₄) dimethyl 1 naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾.

For compounds of the formula NR₁R₂R₃R₄⁽⁺⁾:

(i) none of R₁-R₄ are CH₃; (ii) one of R₁-R₄ is CH₃; (iii) three of R₁-R₄ are CH₃; (iv) all of R₁-R₄ are CH₃; (v) two of R₁-R₄ are CH₃, one of R₁-R₄ is C₆H₅CH₂, and one of R₁-R₄ is an alkyl chain of seven carbon atoms or less; (vi) two of R₁-R₄ are CH₃, one of R₁-R₄ is C₆H₅CH₂, and one of R₁-R₄ is an alkyl chain of nineteen carbon atoms or more; (vii) two of R₁-R₄ are CH₃ and one of R₁-R₄ is the group C₆H₅(CH₂)ₙ, where n>1; (viii) two of R₁-R₄ are CH₃, one of R₁-R₄ is C₆H₅CH₂, and one of R₁-R₄ comprises at least one heteroatom; (ix) two of R₁-R₄ are CH₃, one of R₁-R₄ is C₆H₅CH₂, and one of R₁-R₄ comprises at least one halogen; (x) two of R₁-R₄ are CH₃, one of R₁-R₄ is C₆H₅CH₂, and one of R₁-R₄ comprises at least one cyclic fragment; (xi) two of R₁-R₄ are CH₃ and one of R₁-R₄ is a phenyl ring; or (xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium 14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

In one embodiment of the invention, the preferred one or more surface stabilizers of the invention is any suitable surface stabilizer as described below, with the exclusion of PEG-derivatized vitamin E, which is a non-ionic compound.

In another embodiment of the invention, the preferred one or more surface stabilizers of the invention may be any suitable surface stabilizer as described below, with the exclusion of phospholipids. Finally, in another embodiment of the invention, the preferred one or more surface stabilizers of the invention may be any substance which is categorized by the USFDA as GRAS (“Generally Recognized As Safe”). Preferred surface stabilizers of the invention include, but are not limited to, hypromellose, docusate sodium (DOSS), Plasdone® S630 (random copolymer of vinyl pyrrolidone and vinyl acetate in a 60:40 ratio), hydroxypropyl cellulose SL (HPC-SL), sodium lauryl sulfate (SLS), and combinations thereof. Particularly preferred combinations of surface stabilizers include, but are not limited to, hypromellose and DOSS; Plasdone® S630 and DOSS; HPC-SL and DOSS; and hypromellose, DOSS, and SLS. The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents include, but not limited to: lactose monohydrate, lactose anhydrous, and various starches; examples of binding agents are various celluloses and cross-linked polyvinylpyrrolidone (PVP), microcrystalline cellulose, such as Avicel® PH 101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel. Examples of sweeteners are any natural or artificial sweetener, such as sucrose, xylitol, sucralose, sodium saccharin, cyclamate, aspartame, and acsulfame.

Examples of flavoring agents are Magnasweet® (trademark of MAFCO), bubble gum flavor, and fruit flavors; and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts.

Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Nanoparticulate Fibrate Particle Size

The compositions of the invention comprise nanoparticulate fibrate particles, preferably nanoparticulate fenofibrate particles, which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 990 nm, less than about 980 nm, less than about 970 nm, less than about 960 nm, less than about 950 nm, less than about 940 nm, less than about 930 nm, less than about 920 nm, less than about 910 nm, less than about 900 nm, less than about 890 nm, less than about 880 nm, less than about 870 nm, less than about 860 nm, less than about 850 nm, less than about 840 nm, less than about 830 nm, less than about 820 nm, less than about 810 nm, less than about 800 nm, less than about 790 nm, less than about 780 mm, less than about 770 nm, less than about 760 nm, less than about 750 nm, less than about 740 nm, less than about 730 nm, less than about 720 nm, less than about 710 nm, less than about 700 nm, less than about 690 nm, less than about 680 nm, less than about 670 nm, less than about 660 nm, less than about 650 nm, less than about 640 nm, less than about 630 nm, less than about 620 nm, less than about 610 nm, less than about 600 nm, less than about 590 nm, less than about 580 nm, less than about 570 nm, less than about 560 nm, less than about 550 mm, less than about 540 nm, less than about 530 nm, less than about 520 nm, less than about 510 nm, less than about 500 nm, less than about 490 nm, less than about 480 nm, less than about 470 nm, less than about 460 nm, less than about 450 nm, less than about 440 nm, less than about 430 nm, less than about 420 nm, less than about 410 nm, less than about 400 nm, less than about 390 nm, less than about 380 nm, less than about 370 nm, less than about 360 nm, less than about 350 nm, less than about 340 nm, less than about 330 nm, less than about 320 nm, less than about 310 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100, less than about 75 nm, or less than about 50 nm, as measured by light-scattering methods, microscopy, or other appropriate methods.

By “an effective average particle size of less than about 2000 nm” it is meant that at least 50% of the fibrate (i.e., a “D50”), preferably fenofibrate, particles have a particle size of less than the effective average, by weight or by other suitable measurement techniques (i.e., by volume, number, etc.), i.e., less than about 2000 nm, 1900 nm, 1800 nm, etc., when measured by the above-noted techniques. In other embodiments of the invention, the fibrate particles of the compositions of the invention exist such that at least about 60%, at least about 70%, at least about 80%, at least about 90%, at least about 95% or at least about 99% of the fibrate, preferably fenofibrate, particles have a particle size of less than the effective average as described above, i.e., less than about 2000 nm, 1900 nm, 1800 nm, 1700 nm, . . . less than about 1000 nm, less than about 990 nm, less than about 980 nm, less than about 970 nm, etc. (also referred to as D60, D70, D80, D90, D95, and D99 particle sizes). In another embodiment of the invention, the “effective average particle size” as described above is the mean particle size of the composition (i.e., the invention encompasses a composition having a mean particle size of less than about 2000 nm, . . . less than about 1000 nm, less than about 990 nm, less than about 980 nm, less than about 970 nm, etc.).

In yet another, embodiment of the invention, the mean particle size of the fibrate composition is less than about 100 nm, less than about 75 nm, or less than about 50 nm. In the present invention, the value for D50 of a nanoparticulate fibrate, preferably fenofibrate, composition is the particle size below which 50% of the fibrate particles fall, by weight. Similarly, D90 is the particle size below which 90% of the fibrate particles fall, by weight, volume, number, or any other suitable measurement technique.

5. Concentration of the Fibrate and Surface Stabilizers

The relative amounts of a fibrate, preferably fenofibrate, and one or more surface stabilizers can vary widely. The optimal amount of the individual components can depend, for example, upon the particular fibrate selected, the hydrophilic lipophilic balance (HLB), melting point, and the surface tension of water solutions of the stabilizer, etc. The concentration of the fibrate, preferably fenofibrate, can vary from about 99.5% to about 0.001%, from about 95% to about 0.1%, or from about 90% to about 0.5%, by weight, based on the total combined weight of the fibrate and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the fibrate and at least one surface stabilizer, not including other excipients.

When the quantity of drug is much greater than the quantity of surface stabilizer, such as the 10:1 ratio of drug:surface stabilizer described in the milling examples of U.S. Pat. No. 6,368,620, it can be difficult or impossible to obtain compositions having a narrow particle size distribution curve, or compositions having very small effective average particle sizes, such as D50s of less than 1 micron and D90s of less than 2 microns. Thus, in one embodiment of the invention, the drug:surface stabilizer ratio Less than about 10:1, preferrably 8:1, 7:1, 6:1, most preferrably 5:1, 4:1 and 3:1′,

6. Exemplary Nanoparticulate Fenofibrate Tablet Formulations

Several exemplary fenofibrate tablet formulations of the invention are given below. These examples are not intended to limit the claims in any respect, but rather provide exemplary tablet formulations of fenofibrate of the invention which can be utilized in the methods of the invention. Such exemplary tablets can also comprise a coating agent.

Exemplary Nanoparticulate Fenofibrate Tablet Formulation #1

| Component | g/kg |
|---|---|
| Fenofibrate | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

Exemplary Nanoparticulate Fenofibrate Tablet Formulation #2

| Component | g/kg |
|---|---|
| Fenofibrate | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

Exemplary Nanoparticulate Fenofibrate Tablet Formulation #3

| Component | g/kg |
|---|---|
| Fenofibrate | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

Exemplary Nanoparticulate Fenofibrate Tablet Formulation #4

| Component | g/kg |
|---|---|
| Fenofibrate | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

D. Methods of Making Nanoparticulate Fibrate Compositions

The nanoparticulate fibrate, preferably fenofibrate, compositions can be made using, for example, milling (including but not limited to wet milling), homogenization, precipitation, freezing, template emulsion techniques, supercritical fluid techniques, nano-electrospray techniques, or any combination thereof. Exemplary methods of making nanoparticulate compositions are described in the '684 patent.

Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate fibrate, preferably fenofibrate, compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

In one embodiment of the invention, if heat is utilized during the process of making the nanoparticulate composition, the temperature is kept below the melting point of the fibrate, preferably fenofibrate.

1. Milling to Obtain Nanoparticulate Fibrate Dispersions

Milling a fibrate, preferably fenofibrate, to obtain a nanoparticulate dispersion comprises dispersing the fibrate particles in a liquid dispersion medium in which the fibrate is poorly soluble, followed by applying mechanical means in the presence of grinding media to reduce the particle size of the fibrate to the desired effective average particle size. The grinding media may homogeneous or heterogeneous with respect to media size and composition depending on the desired size range and particle stabilizer(s) selected. The term milling is defined to include any method where there is an input force to a particle system to generate shearing forces within said system resulting in a reduction of the particle size/The dispersion medium can be, for example, water, safflower oil, ethanol, t-butanol, glycerin, polyethylene glycol (PEG), hexane, or glycol. A preferred dispersion medium is water.

The fibrate, preferably fenofibrate, particles can be reduced in size in the presence of at least one surface stabilizer. Alternatively, the fibrate particles can be contacted with one or more surface stabilizers after attrition. Other compounds, such as a diluent, can be added to the fibrate/surface stabilizer composition during the size reduction process. Dispersions can be manufactured continuously or in a batch mode.

In one embodiment of the invention, a mixture of a fibrate and one or more surface stabilizers is heated prior to and/or during the milling process.

2. Precipitation to Obtain Nanoparticulate Fibrate Compositions

Another method of forming the desired nanoparticulate fibrate, preferably fenofibrate, composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities.

Such a method comprises, for example: (1) dissolving a fibrate in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Fibrate Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing particles of a fibrate, preferably fenofibrate, in a liquid dispersion medium, followed by subjecting the dispersion to homogenization to reduce the particle size of the fibrate to the desired effective average particle size. The fibrate particles can be reduced in size in the presence of at least one surface stabilizer. Alternatively, the fibrate particles can be contacted with one or more surface stabilizers either before or after attrition. Other compounds, such as a diluent, can be added to the fenofibrate/surface stabilizer composition either before, during, or after the size reduction process. Dispersions can be manufactured continuously or in a batch mode.

4. Cryogenic Methodologies to Obtain Nanoparticulate Fibrate Compositions

Another method of forming the desired nanoparticulate fibrate compositions is by spray freezing into liquid (“SFL”). This technology comprises an organic or organoaqueous solution of a fibrate, such as fenofibrate, with stabilizers, which is injected into a cryogenic liquid, such as liquid nitrogen. The droplets of the fibrate solution freeze at a rate sufficient to minimize crystallization and particle growth, thus formulating nanostructured fibrate particles. Depending on the choice of solvent system and processing conditions, the nanoparticulate fibrate particles can have varying particle morphology. In the isolation step, the nitrogen and solvent are removed under conditions that avoid agglomeration or ripening of the fibrate particles.

As a complementary technology to SFL, ultra rapid freezing (“URF”) may also be used to created equivalent nanostructured fibrate particles with greatly enhanced surface area. URF comprises an organic or organoaqueous solution of a fibrate with stabilizers onto a cryogenic substrate.

5. Emulsion Methodologies to Obtain Nanoparticulate Fibrate Compositions

Another method of forming the desired nanoparticulate fibrate, such as fenofibrate, composition is by template emulsion. Template emulsion creates nanostructured fibrate particles with controlled particle size distribution and rapid dissolution performance. The method comprises an oil-in-water emulsion that is prepared, then swelled with a non-aqueous solution comprising the fibrate and stabilizers. The particle size distribution of the fibrate particles is a direct result of the size of the emulsion droplets prior to loading with the fibrate a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured fibrate particles are recovered. Various fibrate particles morphologies can be achieved by appropriate control of processing conditions.

6. Supercritical Fluid Techniques Used to Obtain Nanoparticulate Fibrate Compositions

Published International Patent Application No. WO 97/14407 to Pace et al., published Apr. 24, 1997, discloses particles of water insoluble biologically active compounds with an average size of 100 nm to 300 nm that are prepared by dissolving the compound in a solution and then spraying the solution into compressed gas, liquid or supercritical fluid in the presence of appropriate surface modifiers. A “supercritical fluid” is any substance at a temperature and pressure above its thermodynamic critical point. Common examples of supercritical fluids include, but are not limited to, carbon dioxide, ethane, ethylene, propane, propylene, trifluoromethane (fluoroform), chlorotrifluoromethane, trichlorofluoromethane, ammonia, water, cyclohexane, n-pentane and toluene.

7. Nano-Electrospray Techniques Used to Obtain Nanoparticulate Fibrate Compositions

In electrospray ionization a liquid is pushed through a very small charged, usually metal, capillary. This liquid contains the desired substance, e.g., a fibrate such as fenofibrate (or “analyte”), dissolved in a large amount of solvent, which is usually much more volatile than the analyte. Volatile acids, bases or buffers are often added to this solution as well. The analyte exists as an ion in solution either in a protonated form or as an anion. As like charges repel, the liquid pushes itself out of the capillary and forms a mist or an aerosol of small droplets about 10 μm across. This jet of aerosol droplets is at least partially produced by a process involving the formation of a Taylor cone and a jet from the tip of this cone. A neutral carrier gas, such as nitrogen gas, is sometimes used to help nebulize the liquid and to help evaporate the neutral solvent in the small droplets. As the small droplets evaporate, suspended in the air, the charged analyte molecules are forced closer together. The drops become unstable as the similarly charged molecules come closer together and the droplets once again break up. This is referred to as Coulombic fission because it is the repulsive Coulombic forces between charged analyte molecules that drive it. This process repeats itself until the analyte is free of solvent and is a lone ion.

In nanotechnology the electrospray method may be employed to deposit single particles on surfaces, e.g., particles of a fibrate such as fenofibrate. This is accomplished by spraying colloids and making sure that on average there is not more than one particle per droplet. Consequent drying of the surrounding solvent results in an aerosol stream of single particles of the desired type. Here the ionizing property of the process is not crucial for the application but may be put to use in electrostatic precipitation of the particles.

D. Methods of Using the Fibrate Compositions of the Invention

The invention provides a method of rapidly increasing the plasma levels of a fibrate, preferably fenofibrate, in a subject. Such a method comprises orally administering to a subject an effective amount of a composition comprising a fibrate, preferably fenofibrate. The fibrate composition, when tested in fasting subjects in accordance with standard pharmacokinetic practice, produces a maximum blood plasma concentration profile in less than about 6 hours, less than about 5 hours, less than about 4 hours, less than about 3 hours, less than about 2 hours, less than about 1 hour, or less than about 30 minutes after the initial dose of the composition.

The compositions of the invention are useful in treating conditions such as hypercholesterolemia, hypertriglyceridemia, cardiovascular disorders, coronary heart disease, and peripheral vascular disease (including symptomatic carotid artery disease). The compositions of the invention can be used as adjunctive therapy to diet for the reduction of LDL-C, total-C, triglycerides, and Apo B in adult patients with primary hypercholesterolemia or mixed dyslipidemia (Fredrickson Types IIa and IIb). The compositions can also be used as adjunctive therapy to diet for treatment of adult patients with hypertriglyceridemia (Fredrickson Types IV and V hyperlipidemia). Markedly elevated levels of serum tryglycerides (e.g., >2000 mg/dL) may increase the risk of developing pancreatitis. The compositions of the invention can also be used for other indications where lipid regulating agents are typically used. The fenofibrate compositions of the invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

Besides such inert diluents, the composition can also include adjuvants, such as wetting agents, emulsifying and suspending agents, sweetening, flavoring, and perfuming agents.

“Therapeutically effective amount” as used herein with respect to a fibrate, preferably a fenofibrate, dosage shall mean that dosage that provides the specific pharmacological response for which the fibrate is administered in a significant number of subjects in need of such treatment. It is emphasized that “therapeutically effective amount,” administered to a particular subject in a particular instance may not be effective for 100% of patients treated for a specific disease, and will not always be effective in treating the diseases described herein, even though such dosage is deemed a “therapeutically effective amount” by those skilled in the art. It is to be further understood that fibrate dosages are, in particular instances, measured as oral dosages, or with reference to drug levels as measured in blood.

One of ordinary skill will appreciate that effective amounts of a fibrate, such as fenofibrate, can be determined empirically and can be employed in pure form or, where such forms exist, in pharmaceutically acceptable salt, ester, isomer(s), or prodrug form.

Actual dosage levels of a fibrate, such as fenofibrate, in the nanoparticulate compositions of the invention may be varied to obtain an amount of the fibrate that is effective to obtain a desired therapeutic response for a particular composition and method of administration. The selected dosage level therefore depends upon the desired therapeutic effect, the route of administration, the potency of the administered fibrate, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

Several of the formulations in the examples that follow were investigated using a light microscope. Here, “stable” nanoparticulate dispersions (uniform Brownian motion) were readily distinguishable from “aggregated” dispersions (relatively large, non-uniform particles without motion).

Routes of Administration:

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate fibrate, preferably fenofibrate, compositions may also contain adjuvants such as preserving, wetting, emulsifying, and dispersing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the fibrate, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

### EXAMPLE 1

The purpose of this example was to prepare nanoparticulate dispersions of fenofibrate, and to test the prepared compositions for stability in water and in various simulated biological fluids.

Two formulations of fenofibrate were milled, as described in Table 1, by milling the components of the compositions under high energy milling conditions in a DYNO®Mill KDL (Willy A. Bachofen A G, Maschinenfabrik, Basle, Switzerland) for ninety minutes. Formulation 1 comprised 5% (w/w) fenofibrate, 1% (w/w) hypromellose, and 0.05% (w/w) dioctyl sodium sulfosuccinate (DOSS), and Formulation 2 comprised 5% (w/w) fenofibrate, 1% (w/w) Pluronic® S-630 (a random copolymer of vinyl acetate and vinyl pyrrolidone), and 0.05% (w/w) DOSS. The particle size of the resultant compositions was measured using a Horiba LA-910 Laser Scattering Particle Size Distribution Analyzer ((Horiba Instruments, Irvine, Calif.).

TABLE 1
Nanoparticulate Fenofibrate Formulations
Milled Under High Energy Conditions

| Formulation | Drug | Surface Stabilizer | Particle Size |
|---|---|---|---|
| 1 | 5% (w/w) | 1% hypromellose | Mean: 139 nm 90% |
| | | and 0.05% DOSS | <266 nm |
| 2 | 5% (w/w) | 1% S630 and 0.05% | Mean: 233 nm 90% |
| | | DOSS | <355 nm |

Next, the stability of the two formulations was tested in various simulated biological fluids (Table 2) and in water (Table 3) over an extended period of time. For tests in various simulated biological fluids, the composition was deemed stable if the particles remained in a dispersion format with no visible size increase or agglomeration after 30 min. incubation at 40° C. Testing in fluids representing electrolyte fluids is useful as such fluids are representative of physiological conditions found in the human body.

TABLE 2
Stability Testing of Nanoparticulate Fenofibrate Formulations 1 and 2
in Simulated Biological Fluids

| | Electrolyte Test | Electrolyte Test | Electrolyte Test |
|---|---|---|---|
| Formulation | Media #1 | Media #2 | Media #3 |
| 1 | Slight Agglomeration | Acceptable | Acceptable |
| 2 | Heavy | Acceptable | Slight |
| | Agglomeration | | Agglomeration |

TABLE 3
Stability Testing of Nanoparticulate Fenofibrate
Formulations 1 and 2 in Water at 2-8° C.

| Formulation | 3 Days | 1 Week | 2 Weeks | 7 Months |
|---|---|---|---|---|
| 1 | Mean: 149 nm | Mean: 146 nm | Mean: 295 nm | Mean: 1179 nm |
| | 90% <289 nm | 90% <280 nm | 90% <386 nm | 90% <2744 nm |
| 2 | Mean: 824 nm | Mean: 927 nm | Mean: 973 nm | Mean: 1099 nm |
| | 90% <1357 nm | 90% <1476 nm | 90% <1526 nm | 90% <1681 nm |

Stability results indicate that Formulation 1 is preferred over Formulation 2, as Formulation 2 exhibited slight agglomeration in simulated intestinal fluid and unacceptable particle size growth over time.

### EXAMPLE 2

The purpose of this example was to prepare nanoparticulate dispersions of fenofibrate, followed by testing the stability of the compositions in various simulated biological fluids.

Four formulations of fenofibrate were prepared, as described in Table 4, by milling the components of the compositions in a DYNO®-Mill KDL (Willy A. Bachofen A G, Maschinenfabrik, Basle, Switzerland) for ninety minutes.

Formulation 3 comprised 5% (w/w) fenofibrate, 1% (w/w) hydroxypropylcellulose SL (HPC-SL), and 0.01% (w/w) DOSS; Formulation 4 comprised 5% (w/w) fenofibrate, 1% (w/w) hypromellose, and 0.01% (w/w) DOSS; Formulation 5 comprised 5% (w/w) fenofibrate, 1% (w/w) polyvinylpyrrolidone (PVP K29/32), and 0.01% (w/w) DOSS; and Formulation 6 comprised 5% (w/w) fenofibrate, 1% (w/w) Pluronic® S-630, and 0.01% (w/w) DOSS.

The particle size of the resultant compositions was measured using a Horiba LA910 Laser Scattering Particle Size Distribution Analyzer ((Horiba Instruments, Irvine, Calif.).

TABLE 4
Particle Size of Nanoparticulate Fenofibrate Formulations

| Formulation | Drug | Surface Stabilizer | Particle Size |
|---|---|---|---|
| 3 | 5% (w/w) | 1% HPC-SL and | Mean: 696 nm |
| | | 0.01% DOSS | 90% <2086 nm |
| 4 | 5% (w/w) | 1% hypromellose | Mean: 412 nm |
| | | and 0.01% DOSS | 90% <502 nm |
| 5 | 5% (w/w) | 1% PVP and 0.01% | Mean: 4120 nm |
| | | DOSS | 90% <9162 nm |
| 6 | 5% (w/w) | 1% S630 and 0.01% | Mean: 750 nm |
| | | DOSS | 90% <2184 nm |

The results indicate that PVP is not a satisfactory surface stabilizer for fenofibrate, at the particular concentrations of fenofibrate and PVP disclosed, in combination with DOSS, as the mean particle size of Formulation 5 was over two microns. However, PVP may be useful as a surface stabilizer for fenofibrate when it is used alone, in combination with another surface stabilizer, or when different concentrations of PVP and/or fenofibrate are utilized.

Next, the stability of Formulations 4 and 6 was tested in various simulated biological fluids (Table 5).

TABLE 5
Stability Testing of Nanoparticulate Fenofibrate Formulations 3-6
in Simulated Biological Fluids

| | Electrolyte Test | Electrolyte Test | Electrolyte Test |
|---|---|---|---|
| Formulation | Media #1 | Media #2 | Media #3 |
| 3 | N/A | N/A | N/A |
| 4 | Acceptable | Acceptable | Acceptable |
| 5 | N/A | N/A | N/A |
| 6 | Agglomeration | Very slight | Slight agglomeration |
| | | agglomeration | |

The results indicate that Formulation 4, comprising hypromellose and DOSS as surface stabilizers, is preferred as the initial particle size is within the useable range (i.e., 90%<502 nm) and the composition shows no aggregation in various simulated biological fluids.

The next set of examples relates to the redispersibility of the spray granulated powders of the nanoparticulate fenofibrate compositions. The purpose for establishing redispersibility of the spray granulated powder is to determine whether the solid nanoparticulate fenofibrate composition of the invention will redisperse when introduced into biologically relevant media.

### EXAMPLE 3

The purpose of this example was to evaluate the redispersibility of spray granulated powders of preferred nanoparticulate fenofibrate compositions comprising hypromellose and DOSS with or without SLS, a preferred small anionic surfactant. The redispersibility of two powder forms of a spray granulated powder of nanoparticulate fenofibrate was determined, the results of which are shown in Table 6.

TABLE 6

| Physical form | Powder | Powder |
|---|---|---|
| Drug:Sucrose | 1:0.6 | 1:1 |
| Hypromellose:DOSS | 1:0.2 | — |
| Hypromellose:DOSS + SLS | — | 1:0.3 |
| Redispersibility | | |
| DI water | | |
| Mean (nm) | 390 | 182 |
| D90 (nm) | 418 | 260 |
| % <1000 nm | 95.9 | 100.0 |
| Electrolyte Test Medium | | |
| #2 | | |
| Mean (nm) | 258 | 193 |
| D90 (nm) | 374 | 276 |
| % <1000 nm | 99.7 | 100.0 |
| Electrolyte Test Medium | | |
| #3 | | |
| Mean (nm) | 287 | 225 |
| D90 (nm) | 430 | 315 |
| % <1000 nm | 99.6 | 100.0 |

The results show that powders prepared from a granulation feed dispersiontm having hypromellose, DOSS and SLS exhibit excellent redispersiblity.

### EXAMPLE 4

The purpose of this example was to test the redispersibility of a spray granulated powder of nanoparticulate fenofibrate comprising higher levels of DOSS and SLS, as compared to Example 3. The results are shown in Table 7.

TABLE 7

| Physical form | Powder |
|---|---|
| Drug:Sucrose | 1:1 |
| Hypromellose:SLS + DOSS | 1:0.45 |
| Redispersibility | |
| DI water | |
| Mean (nm) | 196 |
| D90 (nm) | 280 |
| % <1000 nm | 100 |
| Electrolyte Test Medium #2 | |
| Mean (nm) | 222 |
| D90 (nm) | 306 |
| % <1000 nm | 100 |
| Electrolyte Test Medium #3 | |
| Mean (nm) | 258 |
| D90 (nm) | 362 |
| % <1000 nm | 100 |

Excellent redispersibility was observed for the tested composition in simulated biological fluids.

### EXAMPLE 5

The purpose of this example was to prepare a nanoparticulate fenofibrate tablet formulation. A fenofibrate nanoparticulate dispersion was prepared by combining the materials listed in Table 8, followed by milling the mixture in a Netzsch LMZ2 Media Mill with Grinding Chamber with a flow rate of 1.0±0.2 LPM and an agitator speed of 3000±100 RPM, utilizing Dow PolyMill™ 500 micron milling media. The resultant mean particle size of the nanoparticulate fenofibrate dispersion (NCD), as measured by a Horiba LA910 Laser Scattering Particle Size Distribution Analyzer ((Horiba Instruments, Irvine, Calif.) was 169 nm.

TABLE 8
Nanoparticulate Fenofibrate Dispersion

| | | |
|---|---|---|
| Fenofibrate | 300 | g/kg |
| Hypromellose, USP (Pharmacoat ® 603) | 60 | g/kg |
| Docusate Sodium, USP | 0.75 | g/kg |
| Purified Water | 639.25 | g/kg |

Next, a granulation feed dispersion (GFD)™ was prepared by combining the nanoparticulate fenofibrate dispersion with the additional components specified in Table 9.

TABLE 9
Nanoparticulate Fenofibrate Granulation
Feed Dispersion

| | | |
|---|---|---|
| Nanoparticulate Fenofibrate Dispersion | 1833.2 | g |
| Sucrose, NF | 550.0 | g |
| Sodium Lauryl Sulfate, NF | 38.5 | g |
| Docusate Sodium, USP/EP | 9.6 | g |
| Purified Water | 723.2 | g |

The fenofibrate GFD was sprayed onto lactose monohydrate (500 g) to form a spray granulated intermediate (SGI) using a Vector Multi-1 Fluid Bed System set to run at the parameters specified in Table 10, below.

TABLE 10
Fluid Bed System Parameters

| | |
|---|---|
| Inlet Air Temperature | 70 ± 10° C. |
| Exhaust/Product Air Temperature | 37 ± 5° C. |
| Air Volume | 30 ± 20 CFM |
| Spray Rate | 15 ± 10 g/min |

The resultant spray granulated intermediate (SGI) of the nanoparticulate fenofibrate is detailed in Table 11, below.

TABLE 11
Spray Granulated Intermediate of the Nanoparticulate
Fenofibrate

| | | |
|---|---|---|
| Fenofibrate NCD | 1833.2 | g |
| Sucrose, NF | 550.0 | g |
| Sodium Lauryl Sulfate, NF | 38.5 | g |
| Docusate Sodium, USP/EP | 9.6 | g |
| Lactose Monohydrate, NF | 500 | g |

The nanoparticulate fenofibrate SGI was then tableted using a Kilian tablet press with a 0.700×0.300″ plain upper and lower caplet shape punches. Each tablet contained 160 mg of fenofibrate. The resulting tablet formulation is shown below in Table 12.

TABLE 12
Nanoparticulate Fenofibrate Tablet
Formulation

| | | |
|---|---|---|
| Nanoparticulate Fenofibrate Spray | 511.0 | mg |
| Granulated Intermediate | | |
| Silicified Microcrystalline Cellulose | 95.0 | mg |
| Crospovidone, NF | 83.0 | mg |
| Magnesium Stearate, NF | 1.0 | mg |

### EXAMPLE 6

The purpose of this example was to assess the effect of food (food effect) on the bioavailability of a nanoparticulate fenofibrate tablet formulation, as prepared in Example 5.

Study Design

A single-dose, three-way cross-over design study, with eighteen subjects, was conducted. The three treatments consisted of: Treatment A: 160 mg nanoparticulate fenofibrate tablet administered under fasted conditions; Treatment B: 160 mg nanoparticulate fenofibrate tablet administered under high fat fed conditions; and Treatment C: 200 mg micronized fenofibrate capsule (TRICOR®) administered under low fat fed conditions.

“Low fat fed” conditions are defined as 30% fat-400 Kcal, and “high fat fed” conditions are defined as 50% fat-1000 Kcal. The length of time between doses in the study was 10 days.

Results

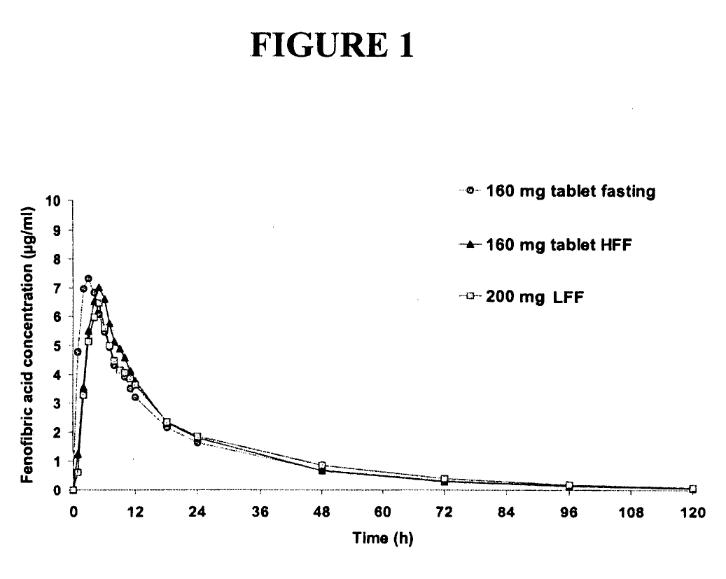

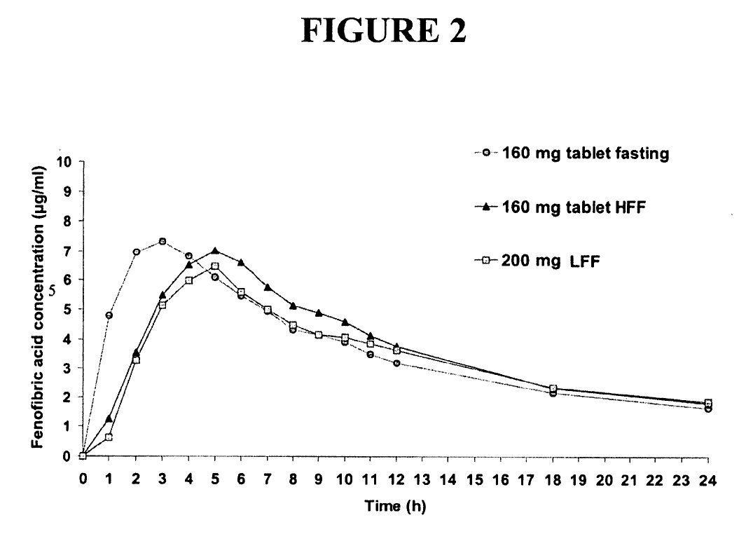

FIG. 1 shows the plasma fenofibric acid profiles (i.e., the fenofibric acid concentration (μg/ml)) over a period of 120 hours for Treatment A, Treatment B, and Treatment C. FIG. 2 shows the same fenofibric acid profiles, but over a 24 hour period rather than a 120 hour period.

Unexpectedly, all three treatments produced approximately the same profile, although the nanoparticulate fenofibrate tablet administered under fasting conditions exhibited a marginally higher maximum fenofibrate concentration. These results are significant for several reasons. First, the nanoparticulate fenofibrate tablet yields substantially similar pharmacokinetic profiles at a lower dosage than that of the conventional microcrystalline fenofibrate capsule: 160 mg vs. 200 mg. A lower dosage is generally seen as beneficial for the patient, as less active agent is administered to the patient. Second, the results show that the nanoparticulate fenofibrate tablet formulation does not exhibit significant differences in absorption when administered in the fed versus the fasted state. This is significant as it eliminates the need for a patient to ensure that they are taking a dose with or without food. Therefore, administration of the nanoparticulate fenofibrate dosage form is expected to result in increased patient compliance. With poor patient compliance, an increase in cardiovascular problems or other conditions for which the fenofibrate is being prescribed could result. The pharmacokinetic parameters of the three tests are shown below in Table 13.

TABLE 13
Pharmacokinetic Parameters
(Mean, Standard Deviation, CV %)

| | Treatment A | Treatment B | Treatment C |
|---|---|---|---|
| AUC (μg/mL · h) | mean = 139.41 | mean = 138.55 | mean = 142.96 |
| | SD = 45.04 | SD = 41.53 | SD = 51.28 |
| | CV % = 32% | CV % = 30% | CV % = 36% |
| Cₘₐₓ (μg/mL) | mean = 8.30 | mean = 7.88 | mean = 7.08 |
| | SD = 1.37 | SD = 1.74 | SD = 1.72 |
| | CV % = 17% | CV % = 22% | CV % = 24% |

The pharmacokinetic parameters first demonstrate that there is no meaningful difference in the amount of drug absorbed when the nanoparticulate fenofibrate tablet is administered in the fed versus the fasted condition (see the AUC results; 139.41 μg/mL·h for the dosage form administered under fasted conditions and 138.55 μg/mL·h for the dosage form administered under fed conditions). Second, the data show that there is no meaningful difference in the rate of drug absorption when the nanoparticulate fenofibrate tablet is administered in the fed versus the fasted condition (see the Cₘₐₓ results; 8.30 μg/mL for the dosage form administered under fasted conditions and 7.88 μg/mL for the dosage form administered under fed conditions). Consequently, the nanoparticulate fenofibrate dosage form eliminates the effect of food on the pharmacokinetics of fenofibrate. Accordingly, the invention encompasses a fibrate composition wherein the pharmacokinetic profile of the fibrate is not affected by the fed or fasted state of a subject ingesting the composition.

Bioequivalence of the Nanoparticulate Fenofibrate Dosage

Form when Administered in the Fed Vs Fasted State

Using the data from Table 13, it was determined whether administration of a nanoparticulate fenofibrate tablet in a fasted state was bioequivalent to administration of a nanoparticulate fenofibrate tablet in a fed state, pursuant to regulatory guidelines. The relevant date from Table 13 is shown below in Table 14, along with the 90% Confidence Intervals (CI). Under U.S. FDA guidelines, two products or treatments are bioequivalent if the 90% CI for AUC and Cₘₐₓ are between 80% and 125%. As shown below in Table 14, the 90% CI for the nanoparticulate fenofibrate fed/fasted methods is 95.2 to 104.3% for AUC and 85.8 to 103.1% for Cₘₐₓ.

TABLE 14
Bioequivalence of Nanoparticulate Fenofibrate Tablet HFF vs.
Nanoparticulate Fenofibrate Tablet Fasted

| | | | CI 90% on log- |
|---|---|---|---|
| | | | transformed |
| | | | data |
| AUC (μg/mL · h) | Nanoparticulate Fenofibrate | 139 | 0.952:1.043 |
| | Tablet 160 mg HFF | | |
| | Nanoparticulate Fenofibrate | 139 | |
| | Tablet 160 mg Fasted | | |
| Cₘₐₓ (μg/mL) | Nanoparticulate Fenofibrate | 7.88 | 0.858:1.031 |
| | Tablet 160 mg HFF | | |
| | Nanoparticulate Fenofibrate | 8.30 | |
| | Tablet 160 mg Fasted | | |

Accordingly, pursuant to regulatory guidelines, administration of a nanoparticulate fenofibrate tablet in the fasted state is bioequivalent to administration of a nanoparticulate fenofibrate tablet in the fed state. Thus, the invention encompasses a fibrate composition wherein administration of the composition to a subject in the fasted state is bioequivalent to administration of the composition to a subject in the fed state.

Moreover, as shown by the data in Table 15 below, administration of a 160 mg nanoparticulate fenofibrate tablet in the fed state is bioequivalent to administration of a 200 mg conventional microcrystalline fenofibrate capsule (TRICOR®) in the fed state. This is because CI 90% for the two treatments falls within 80% to 125% for both AUC and Cₘₐₓ.

TABLE 15
Bioequivalence of Nanoparticulate 160 mg Fenofibrate Tablet HFF
vs. a Microcrystalline 200 mg Fenofibrate Capsule (TRICOR ®) LFF

| | | | CI 90% on log- |
|---|---|---|---|
| | | | transformed data |
| AUC (μg/mL · h) | Nanoparticulate 160 mg | 139 | 0.936:1.026 |
| | Fenofibrate Tablet HFF | | |
| | Microcrystalline 200 mg | 143 | |
| | Fenofibrate Capsule | | |
| | (TRICOR ®) LFF | | |
| Cₘₐₓ (μg/mL) | Nanoparticulate 160 mg | 7.88 | 1.020:1.226 |
| | Fenofibrate Tablet HFF | | |
| | Microcrystalline 200 mg | 7.08 | |
| | Fenofibrate Capsule | | |
| | (TRICOR ®) LFF | | |

The bioequivalence is significant, because it means that the nanoparticulate fenofibrate dosage form exhibits substantially similar drug absorption, but at a lower dose. For the nanoparticulate fenofibrate dosage form to be bioequivalent to the conventional microcrystalline fenofibrate dosage form (e.g., TRICOR®), the dosage form must contain significantly less drug (160 mg vs. 200 mg in the current example). Therefore, the nanoparticulate fenofibrate dosage form significantly increases the bioavailability of the drug.

### EXAMPLE 7

The purpose of this example was to provide nanoparticulate fenofibrate tablet formulations prepared as described in Example 5, above. Shown below in Table 17 is the nanoparticulate fenofibrate dispersion used for making the nanoparticulate fenofibrate tablet formulations.

TABLE 17
Nanoparticulate Fenofibrate Dispersion

| | |
|---|---|
| Fenofibrate | 194.0 g/kg |
| Hypromellose, USP (Pharmacoat ® 603) | 38.81 g/kg |
| Docusate Sodium, USP | 0.485 g/kg |
| Water for injection, USP, EP | 572.7 g/kg |
| Sucrose, NF | 194.0 g/kg |
| Actual Total | 1000.0 g/kg |

Two different tablets were made using the dispersion: a 145 mg nanoparticulate fenofibrate tablet and a 48 mg nanoparticulate fenofibrate tablet. A granulation feed dispersion (GFD) was prepared by combining the nanoparticulate fenofibrate dispersion with sucrose, docusate sodium, and sodium lauryl sulfate.

The fenofibrate GFD was processed and dried in a fluid-bed column (Vector Multi-1 Fluid Bed System), along with lactose monohydrate. The resultant spray granulated intermediate (SGI) was processed through a cone mill, followed by (1) processing in a bin blender with silicified microcrystalline cellulose and crospovidone, and (2) processing in a bin blender with magnesium stearate. The resultant powder was tableted on a rotary tablet press, followed by coating with Opadry® AMB using a pan coater.

Table 18 provides the composition of the 145 mg fenofibrate tablet, and Table 19 provides the composition of the 48 mg fenofibrate tablet.

TABLE 18
145 mg Nanoparticulate Fenofibrate Tablet
Formulation

| Component | g/kg |
|---|---|
| Fenofibrate | 222.54 |
| Hypromellose, USP | 44.506 |
| Docusate Sodium, USP | 4.4378 |
| Sucrose, NF | 222.54 |
| Sodium Lauryl Sulfate, NF | 15.585 |
| Lactose Monohydrate, NF | 202.62 |
| Silicified Microcrystalline Cellulose | 132.03 |
| Crospovidone, NF | 115.89 |
| Magnesium Stearate, NF | 1.3936 |
| Opadry OY-28920 | 38.462 |
| Actual Total | 1000.0 |

TABLE 19
48 mg Nanoparticulate Fenofibrate Tablet Formulation

| Component | g/kg |
|---|---|
| Fenofibrate | 221.05 |
| Hypromellose, USP | 44.209 |
| Docusate Sodium, USP | 4.4082 |
| Sucrose, NF | 221.05 |
| Sodium Lauryl Sulfate, NF | 15.481 |
| Lactose Monohydrate, NF | 201.27 |
| Silicified Microcrystalline Cellulose | 131.14 |
| Crospovidone, NF | 115.12 |
| Magnesium Stearate, NF | 1.3843 |
| Opadry OY-28920 | 44.890 |
| Actual Total | 1000.0 |

### EXAMPLE 8

The purpose of this example is to compare the dissolution of a nanoparticulate 145 mg fenofibrate dosage form according to the invention with a conventional microcrystalline form of fenofibrate (TRICOR®) in a dissolution medium, which is representative of in vivo conditions.

The dissolution of the 145 mg nanoparticulate fenofibrate tablet, prepared in Example 7, was tested in a dissolution medium which is discriminating. Such a dissolution medium is intended to produce different in vitro dissolution profiles for two products having different in vivo dissolution profiles in gastric juices; i.e., the dissolution behavior of the products in the dissolution medium is intended to be predictive of the dissolution behavior within the body. The dissolution medium employed was an aqueous medium containing the surfactant sodium lauryl sulfate at a concentration of 0.025 M. Determination of the amount dissolved was carried out by spectrophotometry, and the tests were repeated 12 times. The rotating blade method (European Pharmacopoeia) was used under the following conditions: volume of medium: 1000 ml; medium temperature: 37° C.; blade rotation speed: 75 RPM; samples taken: every 2.5 minutes;

The results are shown below in Table 20. The table shows individual amounts (%) of drug dissolved at 5, 10, 20, and 30 minutes for twelve different samples, as well as the mean (%) and standard deviation (%) results.

TABLE 20
Dissolution Profile of the Nanoparticulate Fenofibrate 145 mg Table

| Test Sample | 5 min. | 10 min. | 20 min. | 30 min. |
|---|---|---|---|---|
| 1 | 36.1 | 80.9 | 101.7 | 103.6 |
| 2 | 73.4 | 100.5 | 100.1 | 101.8 |
| 3 | 44.0 | 85.6 | 100.0 | 101.4 |
| 4 | 41.0 | 96.1 | 102.3 | 102.5 |
| 5 | 58.7 | 92.9 | 103.4 | 103.5 |
| 6 | 51.9 | 97.8 | 102.6 | 103.4 |
| 7 | 28.6 | 66.9 | 99.3 | 100.4 |
| 8 | 44.7 | 97.4 | 98.8 | 99.3 |
| 9 | 30.1 | 76.9 | 97.0 | 98.0 |
| 10 | 33.6 | 76.8 | 101.8 | 103.5 |
| 11 | 23.5 | 52.6 | 95.8 | 104.0 |
| 12 | 34.6 | 66.9 | 102.8 | 102.2 |
| Mean (%) | 41.7 | 82.6 | 100.5 | 102.0 |
| Standard | 14.1 | 15.2 | 2.4 | 1.9 |
| Deviation (%) | | | | |

U.S. Pat. No. 6,277,405, for “Fenofibrate Pharmaceutical Composition Having High Bioavailability and Method for Preparing It,” describes dissolution of a conventional microcrystalline 160 mg fenofibrate dosage form, e.g., TRICOR®, using the same method described above for the nanoparticulate fenofibrate dosage form (Example 2, cols. 8-9). The results show that the conventional fenofibrate dosage form has a dissolution profile of 10% in 5 min., 20% in 10 min., 50% in 20 min., and 75% in 30 min.

The results show that the nanoparticulate fenofibrate dosage form has significantly more rapid dissolution as compared to the conventional microcrystalline form of fenofibrate. For example, while within 5 minutes approximately 41.7% of the nanoparticulate fenofibrate dosage form is dissolved, only 10% of the microcrystalline TRICOR® dosage form is dissolved. Similarly, while at 10 min. about 82.6% of the nanoparticulate fenofibrate dosage form is dissolved, only about 20% of the microcrystalline TRICOR® dosage form is dissolved during the same time period.

Finally, while at 30 min. principally 100% of the nanoparticulate dosage form is dissolved, only about 75% of the conventional fenofibrate dosage form is dissolved during the same time period.

Thus, the nanoparticulate fenofibrate dosage forms of the invention exhibit significantly improved rates of dissolution.

### EXAMPLE 9

The purpose of this example is to determine whether the bioavailability of a 145 mg nanoparticulate fenofibrate formulation is equivalent to the 200 mg conventional micronized fenofibrate capsule under low-fat meal conditions.

145 mg fenofibrate tablets and 48 mg fenofibrate tablets were prepared as described in Example 7, Tables 18 and 19. This study was a Phase 1, single-dose, open-label study conducted according to a three-period, randomized crossover design. Seventy-two (72) subjects entered the study and were randomly assigned to receive one of three sequences of Regimen A (one 145 mg fenofibrate tablet, test), Regimen B (three 48 mg fenofibrate tablets, test) and Regimen C (one 200 mg fenofibrate capsule, reference) under non-fasting conditions in the morning of Study Day 1 of each period. The sequences of regimens were such that each subject received all three regimens upon completion of the study. Washout intervals of fourteen (14) days separated the doses of the three study periods. Adult male and female subjects in general good health were selected to participate in the study. Subjects were confined to the study site and supervised for approximately six (6) days in each study period. Confinement in each period began in the afternoon on Study Day −1 (1 day prior to the dosing day) and ended after the collection of the 120-hour blood samples and scheduled study procedures were completed on the morning of Study Day 6. With the exception of the breakfast on Study Day 1 in each period, subjects received a standard diet, providing approximately 34% calories from fat per day, for all meals during confinement. On Study Day 1, study subjects received a low-fat breakfast that provided approximately 520 Kcal and 30% of calories from fat beginning 30 minutes prior to dosing. Blood samples were collected from the subjects by venipuncture into 5 mL evacuated collection tubes containing potassium oxalate plus sodium fluoride prior to dosing (0 hours) and at 1, 2, 3, 4, 5, 6, 7, 8, 9, 10, 11, 12, 18, 24, 48, 72, 96 and 120 hours after dosing (Study Day 1) in each period. The blood samples were centrifuged to separate the plasma. The plasma samples were stored frozen until analyzed. Plasma concentrations of fenofibric acid were determined using a validated liquid chromatographic method with mass spectrometric detection.

Values for the pharmacokinetic parameters of fenofibric acid were estimated using noncompartmental methods. First, the maximum observed plasma concentration (Cₘₐₓ) and the time to Cₘₐₓ (peak time, Tₘₐₓ) were determined directly from the plasma concentration-time data. Second, the value of the terminal phase elimination rate constant (λ_{z}) was obtained from the slope of the least squares linear regression of the logarithms of the plasma concentration versus time data from the terminal log-linear phase of the profile. A minimum of three concentration-time data points was used to determine λ_{z}.

The terminal phase elimination half-life (t_{1/2}) was calculated as ln(2)/λ_{z}. Third, the area under the plasma concentration-time curve (AUC) from time 0 to time of the last measurable concentration (AUCₜ) was calculated by the linear trapezoidal rule. The AUC was extrapolated to infinite time by dividing the last measurable plasma concentration (Cₜ) by λ_{z} and adding the quotient to AUCₜ to give AUC_{∞}. Seventy-one (71) subjects completed the study and their data were included in the pharmacokinetic analyses. The pharmocokinetic results are shown in Table 21.

TABLE 21
Pharmacokinetics of Nanoparticulate Fenofibrate

| | Regimen | | |
|---|---|---|---|
| | | | C: One 200 mg |
| Pharmocokinetic | A: One 145 mg | B: Three 48 mg | capsule (reference) |
| Parameters (units) | tablet (test) (n = 71) | tablets (test) (n = 71) | (n = 71) |
| Tₘₐₓ (h) | 3.5 ± 1.2* | 3.6 ± 1.3* | 4.4 ± 1.7 |
| Cₘₐₓ (μg/ml) | 8.80 ± 1.67 | 8.54 ± 1.62 | 8.87 ± 2.29 |
| AUC_{t‡} (μg · h/ml) | 153.5 ± 40.7* | 153.3 ± 41.8* | 174.2 ± 43.6 |
| AUC_{∞‡} (μg · h/ml) | 157.4 ± 44.2* | 157.0 ± 54.1* | 180.4 ± 49.4 |
| t_{1/2¢‡} (h) | 20.7* | 20.1* | 22.0 |

*Statistically significantly different from reference regimen (Regimen C, ANOVA, p < 0.05).
‡ N = 70.
¢ Harmonic mean; evaluation of t_{1/2} were based on statistical test for λ_{z.}

An analysis of variance (ANOVA) was performed for Tₘₐₓ and the natural logarithms of Cₘₐₓ and AUC. The model included effects for cohort, sequence, interaction of cohort and sequence, subject nested within cohort-sequence combination, period, regimen, interaction of cohort and period, and interaction of cohort and regimen. Within the framework of the ANOVA, each test regimen was compared to the reference with a significance level of 0.05 for each individual comparison.

The bioavailability of each test regimen relative to that of the reference regimen was assessed by the two one-sided procedure via 90% confidence intervals. Bioequivalence between a test regimen and the reference regimen was concluded if the 90% confidence intervals from the analyses of the natural logarithms of AUC and Cₘₐₓ were within the 80 to 125% range. The results are shown in Table 22.

TABLE 22
Relative Bioavailability of Nanoparticulate Fenofibrate

| | | 90% Confidence |
|---|---|---|
| Regimens Test vs. Reference | Point Estimate | Interval |
| Test Regimen A vs. Test | 1.008 | 0.968-1.049 |
| Regimen C - for Cₘₐₓ | | |
| Test Regimen A vs. Test | 0.862 | 0.843-0.881 |
| Regimen C - for AUC_{∞} | | |
| Test Regimen B vs. Test | 0.979 | 0.940-1.019 |
| Regimen C for Cₘₐₓ | | |
| Test Regimen B vs. Test | 0.860 | 0.841-0.879 |
| Regimen C for AUC_{∞} | | |

All of the 90% confidence intervals in Table 22 fell within the 80 to 125% range required to document bioequivalence. One 145 mg nanoparticle fenofibrate tablet and three 48 mg nanoparticle fenofibrate tablets were shown to be bioequivalent to one 200 mg conventional micronized fenofibrate capsule.

### EXAMPLE 10

The purpose of this example is to determine whether the bioavailability of a 45 mg nanoparticulate fenofibrate formulation is affected by food. 145 mg nanoparticulate fenofibrate tablets were prepared as described in Example 7, Tables 18 and 19.

This study was a Phase 1, single-dose, open-label study conducted according to a three-period, randomized crossover design. Forty-five (45) subjects entered the study and were randomly assigned to receive one of three sequences of Regimen A (one 145 mg fenofibrate tablet administered under high-fat meal conditions), Regimen B (one 145 mg fenofibrate tablet administered under low fat meal conditions) and Regimen C (one 145 mg fenofibrate tablet administered under fasting conditions). The sequences of regimens were such that each subject received all three regimens upon completion of the study.

Washout intervals of at least fourteen (14) days separated the doses of the three study periods. Adult male and female subjects in general good health were selected to participate in the study.

Subjects were confined to the study site and supervised for approximately 6 days in each study period. Confinement in each period began in the afternoon on Study Day −1 (1 day prior to the dosing day) and ended after the collection of the 120-hour blood samples and scheduled study procedures were completed on the morning of Study Day 6.

On Study Day 1, those subjects assigned to Regimen A received a high-fat breakfast that provided approximately 1000 Kcal and 50% of calories from fat beginning 30 minutes prior to dosing. Those subjects assigned to Regimen B received a low-fat breakfast that provided approximately 520 Kcal and 30% of calories from fat beginning 30 minutes prior to dosing. For those subjects assigned to Regimen C, no food or beverage, except for water to quench thirst, was allowed beginning 10 hours before dosing (Study Day −1) and continuing until after the collection of the 4-hour blood sample on the following day (Study Day 1). All treatments were administered with 240 mL of water. No other fluids were allowed for 1 hour before dosing and 1 hour after dosing. With the exception of the breakfast on Study Day 1 in each period, subjects received a standard well-balanced diet for all meals during confinement. Blood samples were collected from the subjects by venipuncture into 5 mL evacuated collection tubes containing potassium oxalate plus sodium fluoride prior to dosing (0 hours) and at 0.5, 1, 1.5, 2, 3, 4, 5, 6, 7, 8, 9, 10, 11, 12, 18, 24, 48, 72, 96 and 120 hours after dosing (Study Day 1) in each period. The blood samples were centrifuged to separate the plasma. The plasma samples were stored frozen until analyzed. Plasma concentrations of fenofibric acid were determined using a validated liquid chromatographic method with ultraviolet detection.

Values for the pharmacokinetic parameters of fenofibric acid were estimated using noncompartmental methods. First, the maximum observed plasma concentration (Cₘₐₓ) and the time to Cₘₐₓ (peak time, Tₘₐₓ) were determined directly from the plasma concentration-time data. Second, the value of the terminal phase elimination rate constant (λ_{z}) was obtained from the slope of the least squares linear regression of the logarithms of the plasma concentration versus time data from the terminal log-linear phase of the profile. A minimum of three concentration-time data points was used to determine λ_{z}. The terminal phase elimination half-life (t_{1/2}) was calculated as ln(2)/λ_{z}. Third, the area under the plasma concentration-time curve (AUC) from time 0 to time of the last measurable concentration (AUCₜ) was calculated by the linear trapezoidal rule. The AUC was extrapolated to infinite time by dividing the last measurable plasma concentration (Cₜ) by λ_{z} and adding this quotient to AUCₜ to give AUC_{∞}. Forty-four (44) subjects completed the study and were included in the pharmacokinetic analyses. The pharmacokinetic results are shown in Table 23.

TABLE 23
Pharmacokinetics of Nanoparticulate Fenofibrate

| | Regimen | | |
|---|---|---|---|
| Pharmacokinetic | A: High-fat Meal | B: Low-fat Meal | C: Fasting |
| Parameters (units) | (n = 44) | (n = 44) | (n = 44) |
| Tₘₐₓ (h) | 4.27 ± 1.94 | 3.56 ± 1.18 | 2.33 ± 0.73 |
| Cₘₐₓ (μg/ml) | 7.96 ± 1.47 | 7.96 ± 1.43 | 7.94 ± 1.59 |
| AUCₜ (μg · h/ml) | 127.9 ± 35.4 | 123.2 ± 35.0 | 121.6 ± 34.2 |
| AUC_{∞} (μg · h/ml) | 129.9 ± 36.4 | 125.1 ± 35.8 | 123.8 ± 35.7 |
| t_{1/2} (h) | 17.8 ± 4.1 | 18.7 ± 3.7 | 18.9 ± 4.7 |

An analysis of variance (ANOVA) was performed for Tₘₐₓ and the natural logarithms of Cₘₐₓ and AUC. The model included effects for sequence, period, subject nested within sequence and regimen. Within the framework of the ANOVA, each of the high-fat and low-fat meal regimens was compared to the fasting regimen at a significance level of 0.05. There were no statistically significant differences between the sequences and periods. The bioavailability of each test regimen relative to that of the reference regimen was assessed by the two one-sided procedure via 90% confidence intervals. Absence of food effect was concluded if the 90% confidence intervals from the analyses of the natural logarithms of AUC and Cₘₐₓ were within the 80 to 125% bioequivalence range. The absence of food effect is shown in Table 24 for the high-fat meal and in Table 25 for the low-fat meal.

TABLE 24
Nanoparticulate Fenofibrate Food Effect Assessment for a 145 mg
Nanoparticulate Fenofibrate Tablet High-fat Meal versus Fasting

| | | 90% Confidence |
|---|---|---|
| Parameter N = 44 | Point Estimate | Interval |
| AUC∞ | 1.052 | 1.018-1.088 |
| Cₘₐₓ | 1.007 | 0.963-1.054 |

TABLE 25
Nanoparticulate Fenofibrate Food Effect Assessment for a 145 mg
Nanoparticulate Fenofibrate Tablet Low-fat Meal versus Fasting

| | | 90% Confidence |
|---|---|---|
| Parameter N = 44 | Point Estimate | Interval |
| AUC∞ | 1.012 | 0.978-1.046 |
| Cₘₐₓ | 1.009 | 0.964-1.055 |

All of the 90% confidence intervals in Tables 24 and 25 fell within the 80 to 125% bioequivalence range required to document the absence of food effect. Nanoparticle fenofibrate tablets may be administered without regard to meals.

### EXAMPLE 11

The purpose of this example is to determine whether the bioavailability of a 145 mg nanoparticulate fenofibrate formulation is equivalent to the TRICOR® 160 mg conventional micronized fenofibrate tablet under low-fat meal conditions.

145 mg fenofibrate tablets fenofibrate tablets were prepared as described in Example 7, Tables 18. The 160 mg fenofibrate tablets were TRICOR® 160 mg conventional micronized fenofibrate.

This study was a Phase 1, single-dose, open-label study conducted according to a two way, randomized crossover design. Forty (40) subjects entered the study and were randomly assigned to receive one of three sequences of Regimen A (one 145 mg fenofibrate tablet, test), and Regimen B (one 160 mg fenofibrate tablet, reference) under low fat fed conditions in the morning of Study Day 1 of each period. The sequences of regimens were such that each subject received both regimens upon completion of the study. Washout intervals of fourteen (14) days separated the doses of the study periods.

Adult male subjects in general good health were selected to participate in the study. Subjects were confined to the study site and supervised for approximately three (3) days in each study period. Confinement in each period began in the afternoon on Study Day −1 (1 day prior to the dosing day) and ended on Study Day 2 after the collection of the 24-hour blood sample. Subjects returned to the study site for subsequent blood sample collections each morning from Study Day 3 (48 hours after dosing) to Study Day 6 (120 hours after dosing). Scheduled study procedures were completed on the morning of Study Day 6. With the exception of the breakfast on Study Day 1 in each period, subjects received a standard diet for all meals during confinement. On Study Day 1, study subjects received a low-fat breakfast that provided approximately 400 Kcal and 30% of calories from fat. The breakfast was to begin 30 minutes prior to dosing and to be consumed within 25 minutes.

Blood samples were collected from the subjects by venipuncture into 5 mL evacuated collection tubes containing potassium oxalate plus sodium fluoride prior to dosing (0 hours) and at 0.5, 1, 1.5, 2, 3, 4, 5, 6, 7, 8, 9, 10, 11, 12, 18, 24, 48, 72, 96 and 120 hours after dosing (Study Day 1) in each period. The blood samples were centrifuged to separate the plasma. The plasma samples were stored frozen until analyzed. Plasma concentrations of fenofibric acid were determined using a validated high performance liquid chromatographic method with UV detection.

Values for the pharmacokinetic parameters of fenofibric acid were estimated using noncompartmental methods. First, the maximum observed plasma concentration (Cₘₐₓ) and the time to reach Cₘₐₓ (peak time, Tₘₐₓ) were determined directly from the plasma concentration-time data. Second, the value of the terminal phase elimination rate constant (λ_{z}) was obtained from the slope of the least squares linear regression of the logarithms of the plasma concentration versus time data from the terminal log-linear phase of the profile. A minimum of three concentration-time data points was used to determine λ_{z}. The terminal elimination half-life (t_{1/2}) was calculated as ln(2)/λ_{z}. Third, the area under the plasma concentration-time curve (AUC) from time 0 to time of the last quantifiable concentration (AUCₜ) was calculated by the linear trapezoidal rule. The AUC was extrapolated to infinite time by dividing the last measurable plasma concentration (Cₜ) by λ_{z} and adding the quotient to AUCₜ to give AUC_{∞}. Thirty-eight (38) subjects completed the study and their data were included in the pharmacokinetic analyses.

The pharmacokinetic results are shown in Table 26.

TABLE 26
Pharmacokinetics of Nanoparticulate Fenofibrate

Regimen

| Pharmacokinetic | A: One 145 mg tablet | B: One 160 mg tablet |
|---|---|---|
| Parameters (units) | (test) (n = 38) | (reference) (n = 38) |
| Tₘₐₓ (h) | 2.88 ± 1.20 | 3.72 ± 1.15 |
| Cₘₐₓ (μg/ml) | 8.14 ± 1.35 | 6.91 ± 1.60 |
| AUCₜ (μg · h/ml) | 107.99 ± 30.90 | 108.96 ± 31.62 |
| AUC_{∞} (μg · h/ml) | 109.53 ± 31.43 | 110.86 ± 32.13 |
| t_{1/2} (h) | 17.15 ± 3.47 | 18.74 ± 3.73 |

Results are expressed as arithmetic mean ± standard deviation

An analysis of variance (ANOVA) accounting for differences between sequences, periods, subjects within sequence and treatments was performed on log-transformed Cₘₐₓ and AUC.

The two one-sided 90% confidence intervals on log-transformed data for AUC and Cₘₐₓ were used to compare the bioavailability between the test (145 mg fenofibrate tablet) and the reference (TRICOR® 160 mg fenofibrate tablet) treatments. Bioequivalence between the test and the reference treatments under US FDA guidelines was concluded if the 90% confidence intervals were within the 80 to 125% range. The results are shown in Table 27.

TABLE 27
Relative Bioavailability of Nanoparticulate Fenofibrate

| | Point | 90% Confidence |
|---|---|---|
| Regimens Test vs. Reference | Estimate | Interval |
| Test Regimen A vs. Test Regimen B - for | 1.192 | 1.115-1.274 |
| Cₘₐₓ | | |
| Test Regimen A vs. Test Regimen B - for | 0.992 | 0.960-1.026 |
| AUC_{∞} | | |

The 90% confidence interval for the ratio of geometric means for AUC shown in Table 27, fell within the 80 to 125% range required to establish bioequivalence, whereas the 90% confidence interval for the ratio of geometric means for Cₘₐₓ fell slightly outside the 80% to 125% range required to establish bioequivalence (127.4% on the upper side).

It would be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

We claim:

1. A stable fenofibrate composition comprising about 145 mg fenofibrate, wherein:
(a) the fenofibrate is in the form of particles having an effective particle size of less than about 2000 nm;
(b) there is no substantial difference between the AUC of the composition when administered to a human subject under fed versus fasted conditions; and
(c) there is no substantial difference between the Cₘₐₓ of the composition when administered to a human subject under fed as compared to fasted conditions.

2. The composition of claim 1, wherein the composition exhibits bioequivalence upon administration to a human subject in a fed state as compared to administration to a human subject in a fasted state.

3. The composition of claim 2, wherein bioequivalency is established by:
(a) a 90% Confidence Interval for AUC which is between 80% and 125%; and
(b) a 90% Confidence Interval for Cₘₐₓ, which is between 70% and 143%.

4. The composition of claim 2, wherein bioequivalency is established by:
(a) a 90% Confidence Interval for AUC which is between 80% and 125%; and
(b) a 90% Confidence Interval for Cₘₐₓ, which is between 80% and 125%.

5. The composition of claim 1, which is bioequivalent to a microcrystalline TRICOR® 200 mg fenofibrate oral solid dosage form.

6. The composition of claim 1, which is administered as a single daily dose.

7. The composition of claim 1, wherein the difference in AUC of the fenofibrate composition, when administered to a human subject in the fed versus the fasted state, is selected from the group consisting of less than about 35%, less than about 30%, less than about 25%, less than about 20%, less than about 15%, less than about 10%, less than about 5%, and less than about 3%.

8. The composition of claim 1 which exhibits a Tₘₐₓ after administration to fasting human subjects selected from the group consisting of less than about 6 hours, less than about 5 hours, less than about 4 hours, less than about 3 hours, less than about 2 hours, less than about 1 hour, less than about 30 minutes and less than about 15 minutes.

9. The composition of claim 1, wherein in comparative pharmacokinetic testing with a microcrystalline TRICOR® 160 mg tablet or a microcrystalline TRICOR® 200 mg capsule, which are standard commercial formulations of microcrystalline fenofibrate, the composition of claim 1 exhibits a Tₘₐₓ selected from the group consisting of less than about 90%, less than about 80%, less than about 70%, less than about 50%, less than about 30%, and less than about 25% of the Tₘₐₓ exhibited by the microcrystalline TRICOR® tablet or capsule.

10. The composition of claim 1, wherein the fenofibrate is selected from the group consisting of a crystalline phase, an amorphous phase, a semi-crystalline phase, a semi-amorphous phase, and mixtures thereof.

11. The composition of claim 1, wherein the effective average particle size of the particles of fenofibrate is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 990 nm, less than about 980 nm, less than about 970 nm, less than about 960 nm, less than about 950 nm, less than about 940 nm, less than about 930 nm, less than about 920 nm, less than about 910 nm, less than about 900 nm, less than about 890 nm, less than about 880 nm, less than about 870 nm, less than about 860 nm, less than about 850 nm, less than about 840 nm, less than about 830 nm, less than about 820 nm, less than about 810 nm, less than about 800 nm, less than about 790 nm, less than about 780 nm, less than about 770 nm, less than about 760 nm, less than about 750 nm, less than about 740 nm, less than about 730 nm, less than about 720 nm, less than about 710 nm, less than about 700 nm, less than about 690 mm, less than about 680 nm, less than about 670 nm, less than about 660 nm, less than about 650 nm, less than about 640 nm, less than about 630 nm, less than about 620 nm, less than about 610 nm, less than about 600 nm, less than about 590 nm, less than about 580 nm, less than about 570 nm, less than about 560 nm, less than about 550 nm, less than about 540 nm, less than about 530 nm, less than about 520 nm, less than about 510 nm, less than about 500 nm, less than about 490 nm, less than about 480 nm, less than about 470 nm, less than about 460 nm, less than about 450 nm, less than about 440 nm, less than about 430 nm, less than about 420 nm, less than about 410 nm, less than about 400 nm, less than about 390 nm, less than about 380 nm, less than about 370 nm, less than about 360 nm, less than about 350 nm, less than about 340 nm, less than about 330 nm, less than about 320 nm, less than about 310 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100, less than about 75 nm, and less than about 50 nm.

12. The composition of claim 1, wherein the particles of fenofibrate have a particle size distribution in which the D₉₉ is less than about 500 nm.

13. The composition of claim 1, wherein the particles of fenofibrate have a particle size distribution in which the D₅₀ is less than about 350 nm.

14. The composition of claim 1, wherein the particles of fenofibrate have a mean particle size of less than about 100 nm.

15. The composition of claim 1, wherein the composition is formulated:
(a) for administration selected from the group consisting of oral, pulmonary, rectal, opthalmic, colonic, parenteral, intracisternal, intravaginal, intraperitoneal, local, buccal, nasal, otic, and topical administration;
(b) into a dosage form selected from the group consisting of liquid dispersions, oral suspensions, gels, aerosols, ointments, creams, tablets, and capsules;
(c) into a dosage form selected from the group consisting of controlled release formulations, fast melt formulations, lyophilized formulations, delayed release formulations, extended release formulations, pulsatile release formulations, and mixed immediate release and controlled release formulations; or
(d) any combination thereof.

16. The composition of claim 1 further comprising one or more pharmaceutically acceptable excipients, carriers, or a combination thereof.

17. The composition of claim 1, wherein within about 5 minutes at least about 20%, at least about 30%, or at least about 40% of the fenofibrate is dissolved, wherein dissolution is measured in a discriminating aqueous media comprising sodium lauryl sulfate at 0.025 M, and wherein the rotating blade method (European Pharmacopoeia) is used to measure dissolution.

18. The composition of claim 1, wherein within about 10 minutes at least about 40%, at least about 50%, at least about 60%, at least about 70%, or at least about 80% of the fenofibrate is dissolved, wherein dissolution is measured in a discriminating aqueous media comprising sodium lauryl sulfate at 0.025 M, and wherein the rotating blade method (European Pharmacopoeia) is used to measure dissolution.

19. The composition of claim 1, wherein within about 20 minutes at least about 70%, at least about 80%, at least about 90%, or at least about 100% of the fenofibrate is dissolved, wherein dissolution is measured in a discriminating aqueous media comprising sodium lauryl sulfate at 0.025 M, and wherein the rotating blade method (European Pharmacopoeia) is used to measure dissolution.

20. The composition of claim 1, wherein:
(a) within about 5 minutes at least about 30% of the fenofibrate is dissolved;
(b) within about 10 minutes at least about 70% of the fenofibrate is dissolved; and
(c) within about 20 minutes at least about 90% of the fenofibrate is dissolved,
wherein dissolution is measured in a discriminating aqueous media comprising sodium lauryl sulfate at 0.025 M, and wherein the rotating blade method (European Pharmacopoeia) is used to measure dissolution.

21. The composition of claim 1, wherein:
(a) within about 5 minutes at least about 40% of the fenofibrate is dissolved;
(b) within about 10 minutes at least about 80% of the fenofibrate is dissolved; and
(c) within about 20 minutes at least about 100% of the fenofibrate is dissolved,
wherein dissolution is measured in a discriminating aqueous media comprising sodium lauryl sulfate at 0.025 M, and wherein the rotating blade method (European Pharmacopoeia) is used to measure dissolution.

22. The composition of claim 1, wherein upon administration the composition redisperses such that the redispersed particles of fenofibrate have an effective average particle size of less than about 2000 nm.

23. The composition of claim 22, wherein the redispersed particles of fenofibrate have an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 990 nm, less than about 980 nm, less than about 970 nm, less than about 960 nm, less than about 950 nm, less than about 940 nm, less than about 930 nm, less than about 920 nm, less than about 910 nm, less than about 900 nm, less than about 890 nm, less than about 880 nm, less than about 870 nm, less than about 860 nm, less than about 850 nm, less than about 840 nm, less than about 830 nm, less than about 820 nm, less than about 810 nm, less than about 800 nm, less than about 790 nm, less than about 780 nm, less than about 770 nm, less than about 760 nm, less than about 750 nm, less than about 740 nm, less than about 730 nm, less than about 720 nm, less than about 710 nm, less than about 700 nm, less than about 690 nm, less than about 680 nm, less than about 670 nm, less than about 660 nm, less than about 650 nm, less than about 640 nm, less than about 630 nm, less than about 620 nm, less than about 610 nm, less than about 600 nm, less than about 590 nm, less than about 580 nm, less than about 570 nm, less than about 560 nm, less than about 550 nm, less than about 540 nm, less than about 530 nm, less than about 520 nm, less than about 510 nm, less than about 500 nm, less than about 490 nm, less than about 480 mm, less than about 470 nm, less than about 460 nm, less than about 450 nm, less than about 440 nm, less than about 430 nm, less than about 420 nm, less than about 410 nm, less than about 400 nm, less than about 390 nm, less than about 380 mm, less than about 370 nm, less than about 360 nm, less than about 350 nm, less than about 340 nm, less than about 330 nm, less than about 320 nm, less than about 310 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 mm, less than about 150 nm, less than about 140 mm, less than about 130 nm, less than about 120 nm, less than about 110 nm, less than about 100, less than about 75 nm, and less than about 50 nm.

24. The composition of claim 1, wherein the composition redisperses in a biorelevant medium such that the redispersed particles of fenofibrate have an effective average particle size of less than about 2000 nm.

25. The composition of claim 24, wherein the redispersed particles of fenofibrate have an effective average particle size selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 990 nm, less than about 980 nm, less than about 970 nm, less than about 960 nm, less than about 950 nm, less than about 940 nm, less than about 930 nm, less than about 920 nm, less than about 910 nm, less than about 900 nm, less than about 890 nm, less than about 880 nm, less than about 870 nm, less than about 860 nm, less than about 850 nm, less than about 840 nm, less than about 830 nm, less than about 820 nm, less than about 810 nm, less than about 800 nm, less than about 790 nm, less than about 780 nm, less than about 770 nm, less than about 760 nm, less than about 750 nm, less than about 740 nm, less than about 730 nm, less than about 720 nm, less than about 710 nm, less than about 700 nm, less than about 690 nm, less than about 680 nm, less than about 670 nm, less than about 660 nm, less than about 650 nm, less than about 640 nm, less than about 630 nm, less than about 620 nm, less than about 610 nm, less than about 600 nm, less than about 590 nm, less than about 580 nm, less than about 570 nm, less than about 560 nm, less than about 550 nm, less than about 540 nm, less than about 530 nm, less than about 520 nm, less than about 510 nm, less than about 500 nm, less than about 490 nm, less than about 480 nm, less than about 470 nm, less than about 460 nm, less than about 450 nm, less than about 440 nm, less than about 430 nm, less than about 420 nm, less than about 410 nm, less than about 400 nm, less than about 390 nm, less than about 380 nm, less than about 370 nm, less than about 360 nm, less than about 350 nm, less than about 340 nm, less than about 330 nm, less than about 320 nm, less than about 310 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100, less than about 75 nm, and less than about 50 mm.

26. The composition of claim 1, additionally comprising one or more active agents useful in treating one or more conditions selected from the group consisting of dyslipidemia, hyperlipidemia, hypercholesterolemia, and cardiovascular disorders.

27. The composition of claim 1, additionally comprising one or more active agents selected from the group consisting of antihyperglycemic agents, statins, HMG CoA reductase inhibitors, and antihypertensives.

28. The composition of claim 27, wherein the active agent is metformin.

29. The composition of claim 27, wherein the antihypertensive is selected from the group consisting of diuretics, beta blockers, alpha blockers, alpha-beta blockers, sympathetic nerve inhibitors, angiotensin converting enzyme (ACE) inhibitors, calcium channel blockers, angiotensin receptor blockers.

30. The composition of claim 27, wherein the statin or HMG CoA reductase inhibitor is selected from the group consisting of lovastatin; pravastatin; simvastatin; velostatin; atorvastatin, 6-[2-(substituted-pyrrol-1-yl)alkyl]pyran-2-ones, fluvastatin, fluindostatin, pyrazole analogs of mevalonolactone derivatives, rivastatin, pyridyldihydroxyheptenoic acids, 3-substituted pentanedioic acid derivatives, dichloroacetate, imidazole analogs of mevalonolactone, 3-carboxy-2-hydroxy-propane-phosphonic acid derivatives, 2,3-di substituted pyrrole derivatives, 2,3-di-substituted furan derivatives, 2,3-di-substituted thiophene derivatives furan, naphthyl analogs of mevalonolactone, octahydronaphthalenes, keto analogs of mevinolin, phosphinic acid compounds, rosuvastatin, and pitavastatin.

31. The composition of claim 30, wherein the statin or HMG CoA reductase inhibitor is simvastatin.

32. The composition of claim 1, wherein the dosage form is about 10% smaller than the microcrystalline TRICOR® 160 mg tablet.

33. The composition of claim 1, further comprising at least one surface stabilizer.

34. The composition of claim 33, comprising at least one primary surface stabilizer and at least one secondary surface stabilizer.

35. The composition of claim 33, wherein the surface stabilizer is categorized by the U.S. Food and Drug Administration as GRAS.

36. The composition of claim 33, wherein the surface stabilizer is selected from the group consisting of a nonionic surfactant, an anionic surfactant, a cationic surfactant, an ionic surfactant, and a zwitterionic surfactant.

37. The composition of claim 33, wherein at least one surface stabilizer is selected from the group consisting of albumin, human serum albumin, bovine albumin, cetyl pyridinium chloride, gelatin, casein, phosphatides, dextran, glycerol, gum acacia, cholesterol, tragacanth, stearic acid, benzalkonium chloride, calcium stearate, glycerol monostearate, human serum albumin (HSA), HSA derivatives. cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptylβ-D-glucopyranoside; n-heptyl β-D-thioglucoside; n-hexyl β-D-glucopyranoside; nonanoyl-N-methylglucamide; n-noyl β-D-glucopyranoside; octanoyl-Nmethylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, random copolymers of vinyl acetate and vinyl pyrrolidone, cationic polymers, cationic biopolymers, cationic polysaccharides, cationic cellulosics, alginate, cationic nonpolymeric compounds, cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyldimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1 naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

38. The composition of claim 1, further comprising as a surface stabilizer hypromellose, dioctyl sodium sulfosuccinate, sodium lauryl sulfate, or a combination thereof.

39. The composition of claim 38, further comprising sucrose.

40. A fenofibrate composition comprising:
(a) about 145 mg fenofibrate, wherein the fenofibrate is in the form of particles having an effective average particle size of less than about 2000 nm; and
(b) dioctyl sodium sulfosuccinate and hypromellose as surface stabilizers; wherein:
(i) there is no substantial difference between the AUC of the composition when administered to a human subject under fed versus fasted conditions, and
(ii) there is no substantial difference between the Cₘₐₓ of the composition when administered to a human subject under fed as compared to fasted conditions.

41. The composition of claim 40, further comprising sodium lauryl sulfate.

42. The composition of claim 40, further comprising sucrose.

43. The composition of claim 41, further comprising sucrose.

44. The composition of claim 40, wherein administration of the composition to a subject in the fasted state is bioequivalent to administration of the composition to a subject in the fed state.

45. The composition of claim 44, wherein bioequivalency is established by:
(a) a 90% Confidence Interval for AUC which is between 80% and 125%, and
(b) a 90% Confidence Interval for Cₘₐₓ, which is between 70% and 143%.

46. The composition of claim 44, wherein bioequivalency is established by:
(a) a 90% Confidence Interval for AUC which is between 80% and 125%, and
(b) a 90% Confidence Interval for Cₘₐₓ, which is between 80% and 125%.

47. A method of treating a subject in need comprising administering to the subject a therapeutically effective amount of a composition comprising about 145 mg fenofibrate, wherein the fenofibrate is in the form of particles having an effective average particle size of less than about 2000 nm, wherein:
(a) there is no substantial difference between the AUC of the composition when administered to a human subject under fed versus fasted conditions, and
(b) there is no substantial difference between the Cₘₐₓ of the composition when administered to a human subject under fed as compared to fasted conditions.

48. The method of claim 47, wherein administration of the composition to a subject in a fasted state is bioequivalent to administration of the composition to a subject in a fed state.

49. The method of claim 48, wherein bioequivalency is established by:
(a) a 90% Confidence Interval for AUC which is between 80% and 125%, and
(b) a 90% Confidence Interval for Cₘₐₓ, which is between 70% and 143%.

50. The method of claim 48, wherein bioequivalency is established by:
(a) a 90% Confidence Interval for AUC which is between 80% and 125%, and
(b) a 90% Confidence Interval for Cₘₐₓ, which is between 80% and 125%.

51. The method of claim 47, wherein the method is used to treat a condition selected from the group consisting of hypercholesterolemia, hypertriglyceridemia, coronary heart disease, cardiovascular disorders, and peripheral vascular disease.

52. The method of claim 47, wherein the method is used as adjunctive therapy to diet for the reduction of LDL-C, total-C, triglycerides, or Apo B in adult patients with primary hypercholesterolemia or mixed dyslipidemia.

53. The method of claim 47, wherein the method is used as adjunctive therapy to diet for treatment of adult patients with hypertriglyceridemia.

54. The method of claim 47, wherein the method is used to decrease the risk of pancreatitis.

55. The method of claim 47, wherein the method is used to treat indications where lipid regulating agents are typically used.

56. The method of claim 47, wherein the composition additionally comprises one or more active agents useful in treating one or more conditions selected from the group consisting of dyslipidemia, hyperlipidemia, hypercholesterolemia, and cardiovascular disorders.

57. The method of claim 47, wherein the composition additionally comprises one or more active agents selected from the group consisting of antihyperglycemia agents, statins, HMG CoA reductase inhibitors, and antihypertensives.

58. The method of claim 57, wherein the active agent is metformin.

59. The method of claim 57, wherein the antihypertensive is selected from the group consisting of diuretics, beta blockers, alpha blockers, alpha-beta blockers, sympathetic nerve inhibitors, angiotensin converting enzyme (ACE) inhibitors, calcium channel blockers, angiotensin receptor blockers.

60. The method of claim 57, wherein the statin or HMG CoA reductase inhibitor is selected from the group consisting of lovastatin; pravastatin; simvastatin; velostatin; atorvastatin, 6-[2-(substituted-pyrrol-1-yl)alkyl]pyran-2-ones, fluvastatin, fluindostatin, pyrazole analogs of mevalonolactone derivatives, rivastatin, pyridyldihydroxyheptenoic acids, 3-substituted pentanedioic acid derivatives, dichloroacetate, imidazole analogs of mevalonolactone, 3-carboxy-2-hydroxy-propane-phosphonic acid derivatives, 2,3-di-substituted pyrrole derivatives, 2,3-di-substituted furan derivatives, 2,3-di-substituted thiophene derivatives furan, naphthyl analogs of mevalonolactone, octahydronaphthalenes, keto analogs of mevinolin, and phosphinic acid compounds.

61. The method of claim 60, wherein the statin or HMG CoA reductase inhibitor is simvastatin.

62. A stable fenofibrate composition for oral administration comprising:
(a) particles of fenofibrate having an effective average particle size of less than about 2 microns; and
(b) at least one surface stabilizer,
wherein the composition does not contain any trace residues of a supercritical fluid.

63. A stable fenofibrate composition for oral administration comprising:
(a) particles of fenofibrate having an effective average particle size of less than about 2 microns; and
(b) at least one surface stabilizer,
wherein the composition has a narrow particle size distribution.
